SEQUENCE LISTING (13 sequences)

SEQ ID NO: 1 - protein, 19 aa, Homo sapiens
  MOD_RES (4)..(4): hydroxyproline
SGMPGPPGIPGPPGPPGVP

SEQ ID NO: 2 - protein, 23 aa, Homo sapiens
  MOD_RES (9)..(9): hydroxyproline
GDAGPVGPPGPPGPPGPPGPPSA

SEQ ID NO: 3 - protein, 19 aa, Homo sapiens
  MOD_RES (5)..(5): hydroxyproline
PVGPPGPPGPPGPPGPPSA

SEQ ID NO: 4 - protein, 18 aa, Homo sapiens
  MOD_RES (4)..(4): hydroxyproline
AGPPGMPGPRGSPGPQGV

SEQ ID NO: 5 - protein, 17 aa, Homo sapiens
GSVIDQSRVLNLGPITR

SEQ ID NO: 6 - protein, 16 aa, Homo sapiens
VIDQSRVLNLGPITRK

SEQ ID NO: 7 - protein, 16 aa, Homo sapiens
GSVIDQSRVLNLGPIT

SEQ ID NO: 8 - protein, 14 aa, Homo sapiens
VIDQSRVLNLGPIT

SEQ ID NO: 9 - protein, 20 aa, Homo sapiens
  MOD_RES (8)..(8): hydroxyproline
RVGLPGPPGPPGPPGKPGQD

SEQ ID NO: 10 - protein, 19 aa, Homo sapiens
  MOD_RES (7)..(7): hydroxyproline
SGDPGPPGPPGKEGLPGPQ

SEQ ID NO: 11 - protein, 26 aa, Homo sapiens
  MOD_RES (19)..(19): hydroxyproline
SPEGGGGAGARGGAWPTAPAPLPPAT

SEQ ID NO: 12 - protein, 18 aa, Homo sapiens
  MOD_RES (8)..(8): hydroxyproline
QLIVEPGPPGPPGPPGPM

SEQ ID NO: 13 - protein, 22 aa, Homo sapiens
  MOD_RES (8)..(8): hydroxyproline
TPGERGPPGPPGPPGPPGPPAP

(19) United States
(12) Patent Application Publication
(10) Pub. No.: US 2013/0316921 A1
(43) Pub. Date: Nov. 28, 2013

(54) METHODS FOR DIAGNOSIS OF KAWASAKI DISEASE

(71) Applicants: THE REGENTS OF THE UNIVERSITY OF CALIFORNIA, Oakland, CA (US); THE BOARD OF TRUSTEES OF THE LELAND STANFORD JUNIOR UNIVERSITY, Palo Alto, CA (US)

(72) Inventors: Harvey J. Cohen, Los Altos, CA (US); Jane C. Burns, La Jolla, CA (US); John C. Whitin, Palo Alto, CA (US); Bruce Xuefeng Ling, Palo Alto, CA (US); James Schilling, San Mateo, CA (US)

(73) Assignees: THE REGENTS OF THE UNIVERSITY OF CALIFORNIA, Oakland, CA (US); THE BOARD OF TRUSTEES OF THE LELAND STANFORD JUNIOR UNIVERSITY, Palo Alto, CA (US)

(21) Appl. No.: 13/910,817

(22) Filed: Jun. 5, 2013

Related U.S. Application Data
(63) Continuation of application No. PCT/US2012/023739, filed on Feb. 3, 2012.
(60) Provisional application No. 61/567,321, filed on Dec. 6, 2011.
(60) Provisional application No. 61/444,735, filed on Feb. 20, 2011.

(51) Int. Cl. C12Q 1/68; G01N 33/68

(52) U.S. Cl. CPC C12Q 1/6883; G01N 33/6893
USPC 506/7; 435/7.92; 436/501; 435/6.12; 435/6.11

(57) ABSTRACT

Methods for diagnosis of Kawasaki disease (KD) are disclosed. In particular, the invention relates to the use of biomarkers for aiding diagnosis, prognosis, and treatment of KD, and more specifically to biomarkers that can be used to distinguish KD from other inflammatory diseases, including infectious illness and acute febrile illness.

### CROSS-REFERENCE TO RELATED APPLICATIONS

This application is a 35 U.S.C. §111(a) continuation of PCT international application number PCT/US2012/023739 filed on Feb. 3, 2012, incorporated herein by reference in its entirety, which is a nonprovisional of U.S. provisional patent application Ser. No. 61/444,735 filed on Feb. 20, 2011, incorporated herein by reference in its entirety, and a nonprovisional of U.S. provisional patent application Ser. No. 61/567,321 filed on Dec. 6, 2011, incorporated herein by reference in its entirety. Priority is claimed to each of the foregoing applications.

The above-referenced PCT international application was published as PCT International Publication No. WO 2012/112315 on Aug. 23, 2012, incorporated herein by reference in its entirety.

### STATEMENT REGARDING FEDERALLY SPONSORED RESEARCH OR DEVELOPMENT

This invention was made with Government support under contracts R21 HL086835, RO1 HL69413, and K24-HL074864 awarded by the National Institutes of Health. The Government has certain rights in this invention.

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

The present invention pertains generally to methods for diagnosis of Kawasaki disease (KD). In particular, the invention relates to the use of biomarkers for aiding diagnosis, prognosis, and treatment of KD, and more specifically to biomarkers that can be used to distinguish KD from other inflammatory diseases, including infectious illness and acute febrile illness.

2. Description of Related Art

Kawasaki disease (KD) is an acute vasculitis affecting infants and children and the leading cause of acquired pediatric heart disease in the U.S. and Japan (Burns (2009) Indian J. Pediatr. 76:71-76). The cause of KD remains unknown, though epidemiologic and clinical observations indicate that the inflammatory process may be triggered by a viral infection (Gedalia (2007) Curr. Rheumatol. Rep. 9:336-341). KD is currently diagnosed based on clinical observations and supportive non-specific laboratory tests (Kawasaki et al. (1974) Pediatrics 54:271-276; Morens et al. (1978) Hosp. Pract. 13:109-112, 119-120). There is, however, no specific diagnostic test, and it can be difficult to discriminate KD from other inflammatory diseases and febrile illnesses. If not diagnosed and treated promptly, patients with KD may develop coronary artery dilatation or aneurysms. The cardiovascular damage can largely be prevented by timely administration of intravenous immunoglobulin (IVIG). Thus, there remains a need for sensitive and specific diagnostic tests for KD that can discriminate KD from other inflammatory diseases and febrile illnesses and enable early treatment of the disease to prevent cardiovascular damage.

### BRIEF SUMMARY OF THE INVENTION

The invention relates to the use of biomarkers for diagnosis of KD. In particular, the inventors have discovered panels of biomarkers whose expression profiles can be used to diagnose KD and to distinguish KD from other inflammatory diseases, including infectious illness and acute febrile illness. These biomarkers can be used alone or in combination with one or more additional biomarkers or relevant clinical parameters in prognosis, diagnosis, or monitoring treatment of KD.

In one aspect, the invention includes a method for diagnosing KD in a subject. The method comprises (i) measuring the level of a plurality of biomarkers in a biological sample derived from a subject; and (ii) analyzing the levels of the biomarkers and comparing with respective reference value ranges for the biomarkers, wherein differential expression of one or more biomarkers in the biological sample compared to one or more biomarkers in a control sample obtained from a healthy individual, who does not have KD, indicates that the subject has KD.

In certain embodiments, the level of one or more biomarkers is compared with reference value ranges for the biomarkers. The reference value ranges can represent the level of one or more biomarkers found in one or more samples of one or more subjects without KD (i.e., normal samples). Alternatively, the reference values can represent the level of one or more biomarkers found in one or more samples of one or more subjects with KD.

Biomarkers that can be used in the practice of the invention include polypeptides comprising amino acid sequences from proteins including, but not limited to, collagen type 16 alpha 1 (COL16A1), collagen type 1 alpha 1 (COL1A1), collagen type 3 alpha 1 (COL3A1), uromodulin (UMOD), collagen type 9 alpha 3 (COL9A3), collagen type 23 alpha 1 (COL23A1), collectin sub-family member 12 (COLEC12), unnamed protein product Q6ZSL6 (Q6ZSL6), and EMI domain containing 1 (EMID1); and peptide fragments thereof; and polynucleotides comprising nucleotide sequences from genes or RNA transcripts of genes, including but not limited to, TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH. In one embodiment, the biomarker is a peptide comprising an amino acid sequence selected from the group consisting of SEQ ID NOS:1-13, or comprising an amino acid sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, a panel of biomarkers is used for diagnosis of KD. Biomarker panels of any size can be used in the practice of the invention. Biomarker panels for diagnosing KD typically comprise at least 4 biomarkers and up to 30 biomarkers, including any number of biomarkers in between, such as 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the invention includes a biomarker panel comprising at least 4, or at least 5, or at least 6, or at least 7, or at least 8, or at least 9, or at least 10 or more biomarkers. Although smaller biomarker panels are usually more economical, larger biomarker panels (i.e., greater than 30 biomarkers) have the advantage of providing more detailed information and can also be used in the practice of the invention.

In certain embodiments, a panel of biomarkers comprising one or more COL16A1, COL1A1, COL3A1, UMOD, COL9A3, COL23A1, COLEC12, Q6ZSL6, and EMID1 polypeptides or peptide fragments thereof is used for diagnosis of KD. In one embodiment, the panel of biomarkers comprises one or more peptides comprising an amino acid sequence selected from the group consisting of SEQ ID NOS: 1-13, or comprising an amino acid sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto.

In certain embodiments, a panel of biomarkers comprising one or more TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides is used for diagnosis of KD.

Biomarker polypeptides can be measured, for example, by performing an enzyme-linked immunosorbent assay (ELISA), a radioimmunoassay (RIA), an immunofluorescent assay (IFA), immunohistochemistry (IHC), a sandwich assay, magnetic capture, microsphere capture, a Western Blot, surface enhanced Raman spectroscopy (SERS), flow cytometry, or mass spectrometry. In certain embodiments, the level of a biomarker is measured by contacting an antibody with the biomarker, wherein the antibody specifically binds to the biomarker, or a fragment thereof containing an antigenic determinant of the biomarker. Antibodies that can be used in the practice of the invention include, but are not limited to, monoclonal antibodies, polyclonal antibodies, chimeric antibodies, recombinant fragments of antibodies, Fab fragments, Fab′ fragments, F(ab′)₂ fragments, Fᵥ fragments, or sa_{y} fragments.

Biomarker polynucleotides (e.g., coding transcripts) can be detected, for example, by microarray analysis, polymerase chain reaction (PCR), reverse transcriptase (RT-PCR), Northern blot, or serial analysis of gene expression (SAGE).

In certain embodiments, clinical parameters are used for diagnosis of KD, either alone or in combination with the biomarkers described herein. In one embodiment, the invention includes a method for determining a clinical score for a subject suspected of having KD. The method comprises measuring at least seven clinical parameters for the subject, including duration of fever, concentration of hemoglobin in the blood, concentration of C-reactive protein in blood, white blood cell count, percent eosinophils in the blood, percent monocytes in the blood, and percent immature neutrophils in the blood. A clinical score can be calculated using, e.g., multivariate linear discriminant analysis (LDA) from the values of the clinical parameters. The clinical score can then be classified as a low risk KD clinical score, an intermediate risk KD clinical score, or a high risk KD clinical score by methods described herein.

In one embodiment, the invention includes a method for diagnosing KD in a subject comprising (i) determining a KD clinical score for the subject; and (ii) measuring the level of a plurality of biomarkers in a biological sample derived from the subject; and analyzing the levels of the biomarkers and comparing with respective reference value ranges for the biomarkers. A panel of biomarkers comprising one or more COL16A1, COL1A1, COL3A1, UMOD, COL9A3, COL23A1, COLEC12, Q6ZSL6, and EMID1 polypeptides, or peptide fragments thereof, may be used in combination with the clinical score for diagnosis of KD. In one embodiment, the panel of biomarkers comprises one or more polypeptides comprising sequences selected from the group consisting of SEQ ID NOS:1-13.

Alternatively or in addition, a panel of biomarkers comprising one or more TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides can be used for diagnosis of KD.

Methods of the invention, as described herein, can be used to distinguish a diagnosis of KD for a subject from infectious illness or acute febrile illness. A low KD clinical score indicates that a patient is unlikely to have KD, whereas a high KD clinical score indicates that a patient is highly likely to have KD. An intermediate KD clinical score for a subject can be used in combination with a biomarker expression profile for the subject to distinguish KD from infectious illness or acute febrile illness. In one embodiment, an intermediate KD clinical score is used in combination with the expression profile of a panel of biomarkers comprising one or more COL16A1, COL1A1, COL3A1, UMOD, COL9A3, COL23A1, COLEC12, Q6ZSL6, and EMID1 polypeptides; or peptide fragments thereof, in diagnosis of a patient. In another embodiment, an intermediate KD clinical score is used in combination with the expression profile of a panel of biomarkers comprising one or more TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides in diagnosis of a patient. In yet another embodiment, an intermediate KD clinical score is used in combination with the expression profiles from two panels of biomarkers, wherein the first panel of biomarkers comprises COL16A1, COL1A1, COL3A1, UMOD, COL9A3, COL23A1, COLEC12, Q6ZSL6, and EMID1 polypeptides or peptide fragments thereof; and the second panel of biomarkers comprises TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides.

In certain embodiments, patient data is analyzed by one or more methods including, but not limited to, multivariate linear discriminant analysis (LDA), receiver operating characteristic (ROC) analysis, ensemble data mining methods, cell specific significance analysis of microarrays (csSAM), and multi-dimensional protein identification technology (MUDPIT) analysis.

In another aspect, the invention includes a biomarker panel comprising a plurality of biomarkers for diagnosing KD, wherein one or more biomarkers are selected from the group consisting of COL16A1, COL1A1, COL3A1, UMOD, COL9A3, COL23A1, COLEC12, Q6ZSL6, and EMID1 polypeptides; and peptide fragments thereof, and TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides. In one embodiment, the invention includes a biomarker panel comprising COL16A1, COL1A1, COL3A1, UMOD, COL9A3, COL23A1, COLEC12, Q6ZSL6, and EMID1 polypeptides; or peptide fragments thereof. An exemplary biomarker panel comprises peptides consisting of sequences selected from the group consisting of SEQ ID NOS:1-13, or comprising sequences displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto. In another embodiment, the invention includes a biomarker panel comprising TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides.

In another embodiment, the invention includes a method for evaluating the effect of an agent for treating KD in a subject, the method comprising: analyzing the level of each of one or more KD biomarkers in biological samples derived from the subject before and after the subject is treated with the agent, and comparing the levels of the biomarkers with respective reference value ranges for the biomarkers.

In another embodiment, the invention includes a method for monitoring the efficacy of a therapy for treating KD in a subject, the method comprising: analyzing the level of each of one or more KD biomarkers in biological samples derived from the subject before and after the subject undergoes the therapy, and comparing the levels of the biomarkers with respective reference value ranges for the biomarkers.

In another embodiment, the invention includes a method of selecting a patient suspected of having KD for treatment with an intravenous immunoglobulin (WIG), the method comprising: (i) determining the KD clinical score of the patient, and (ii) selecting the patient for treatment with IVIG if the patient has a KD clinical score in the high risk range, or a KD clinical score in the intermediate risk range and a positive KD diagnosis based on the expression profile of one or more biomarker panels described herein.

In another aspect, the invention includes a diagnostic system comprising a storage component (i.e., memory) for storing data, wherein the storage component has instructions for determining the diagnosis of the subject stored therein; a computer processor for processing data, wherein the computer processor is coupled to the storage component and configured to execute the instructions stored in the storage component in order to receive patient data and analyze patient data according to an algorithm; and a display component for displaying information regarding the diagnosis of the patient. The storage component may include instructions for performing multivariate linear discriminant analysis (LDA), receiver operating characteristic (ROC) analysis, ensemble data mining methods, cell specific significance analysis of microarrays (csSAM), and multi-dimensional protein identification technology (MUDPIT) analysis, as described herein (see Example 1). The storage component may further include instructions for performing a sequential diagnosis, as described herein (see Example 1).

In certain embodiments, the invention includes a computer implemented method for diagnosing a patient suspected of having KD, the computer performing steps comprising: receiving inputted patient data; calculating a clinical score for the patient; classifying the clinical score as a low risk KD clinical score, an intermediate risk KD clinical score, or a high risk KD clinical score; analyzing the level of a plurality of biomarkers and comparing with respective reference value ranges for the biomarkers; calculating the likelihood that the patient has KD; and displaying information regarding the diagnosis of the patient.

In one embodiment, the inputted patient data comprises at least 7 clinical parameters selected from the group consisting of duration of fever, concentration of hemoglobin in blood, concentration of C-reactive protein in blood, white blood cell count, percent eosinophils in blood, percent monocytes in blood, and percent immature neutrophils in blood. The inputted patient data may further comprise values for the levels of one or more biomarkers in a biological sample from the patient. For example, the inputted patient data may further comprise values for the levels of one or more biomarkers selected from the group consisting of a COL16A1 polypeptide, a COL1A1 polypeptide, a COL3A1 polypeptide, a UMOD polypeptide, a COL9A3 polypeptide, a COL23A1 polypeptide, a COLEC12 polypeptide, a Q6ZSL6 polypeptide, and an EMID1 polypeptide; and peptide fragments thereof. Alternatively or in addition, the inputted patient data may further comprise values for the levels of one or more biomarkers in a biological sample from the patient, wherein the biomarkers are selected from the group consisting of a TLR7 polynucleotide, a CXCL10 polynucleotide, a LMO2 polynucleotide, a PLXDC1 polynucleotide, a MARCH1 polynucleotide, a IFI30 polynucleotide, a LYN polynucleotide, a CDC42EP2 polynucleotide, a MS4A14 polynucleotide, a PARP14 polynucleotide, a RAC2 polynucleotide, a SRF polynucleotide, a NKTR polynucleotide, a LAP3 polynucleotide, a APOL3 polynucleotide, a STAT1 polynucleotide, a GCNT1 polynucleotide, a CAMK4 polynucleotide, a MRPS25 polynucleotide, a P2RY8 polynucleotide, a ADD3 polynucleotide, a TRIM26 polynucleotide, a ARRB1 polynucleotide, GNAS, a ISG20 polynucleotide, PCGF5, a PRPF18 polynucleotide, a CRTAM polynucleotide, a LHPP polynucleotide, a RASGRP 1 polynucleotide, a CMPK2 polynucleotide, and an RHOH polynucleotide.

In another aspect, the invention includes a kit for diagnosing KD in a subject. The kit may include a container for holding a biological sample isolated from a human subject suspected of having KD, at least one agent that specifically detects a KD biomarker; and printed instructions for reacting the agent with the biological sample or a portion of the biological sample to detect the presence or amount of at least one KD biomarker in the biological sample. The agents may be packaged in separate containers. The kit may further comprise one or more control reference samples and reagents for performing an immunoassay and/or microarray analysis for detection of biomarkers as described herein.

In certain embodiments, the kit includes agents for detecting polypeptides and/or polynucleotides of a biomarker panel comprising a plurality of biomarkers for diagnosing KD, wherein one or more biomarkers are selected from the group consisting of COL16A1, COL1A1, COL3A1, UMOD, COL9A3, COL23A1, COLEC12, Q6ZSL6, and EMID1 polypeptides; and peptide fragments thereof, and TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides. In one embodiment, the kit includes agents for detecting biomarkers of a biomarker panel comprising COL16A1, COL1A1, COL3A1, UMOD, COL9A3, COL23A1, COLEC12, Q6ZSL6, and EMID1 polypeptides, or peptide fragments thereof. For example, the kit may include agents for detecting peptides of a biomarker panel comprising peptides comprising sequences selected from the group consisting of SEQ ID NOS:1-13, or sequences displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto. In another embodiment, the kit includes agents for detecting polynucleotides of a biomarker panel comprising TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides. Furthermore, the kit may include agents for detecting more than one biomarker panel, such as two or three biomarker panels, which can be used alone or together in any combination, and/or in combination with clinical parameters for diagnosis of KD.

These and other embodiments of the subject invention will readily occur to those of skill in the art in view of the disclosure herein.

### BRIEF DESCRIPTION OF THE SEVERAL VIEWS OF THE DRAWING(S)

The invention will be more fully understood by reference to the following drawings which are for illustrative purposes only:

FIG. 1 shows a linear discriminant analysis of KD training cohort patients, including the calculated coefficients of linear discriminants (LD1) of each assayed clinical parameter, a modified two by two contingency table showing the percentage of classifications that agreed with the clinical diagnosis, and density plots of the clinical score distribution of both KD and FC patients (KD, dark gray density plot; FC, light gray density plot). We stratified all the KD and FC patients, using the clinical scores, into low, intermediate and high groups, where the group boundaries were decided by the diagnosis with 95% accuracy (two dotted vertical lines).

FIGS. 2A and 2B show a cell type-specific significant analysis of KD and FC whole blood microarray data. FIG. 2A shows a SAM analysis revealing no differentially expressed genes in whole blood. FIG. 2B shows a csSAM analysis revealing differential expression in lymphocytes, but not in other cell types (Up: up regulated in FC; Down: down regulated in FC; Y axis: false discovery rate (FDR); X axis: number of differential genes at a given FDR).

FIG. 3 shows a urine peptidome analysis. The top panel summarizes the MUDPIT discovery and MALDI mass spectrometry confirmation processes. The bottom table lists the 13 confirmed urine peptide biomarkers (SEQ ID NOS:1-13) discriminating KD and FC (M/Z: mass to charge ratio, SC: spectral counting difference, post translation modifications: * hydroxylation; # methionine oxidation. U Test: P-value).

FIG. 4 shows a sequential predictive algorithm integrating both the clinical and molecular biomarker findings to improve KD diagnosis (KD, dark gray; FC, light gray). The left panel shows all three cohort samples, clinical training, testing 1 and 2, which were stratified and ordered according to their clinical scores. Patients with intermediate scores were further analyzed by 32 gene or 13 urine peptide based classifiers to discriminate KD and FC. The right panel shows a ROC analysis of all three-cohort patients with intermediate clinical scores analyzed by clinical score (black), cell-specific, whole blood gene-based (medium gray) and urine peptide-based (light gray) classifiers.

FIG. 5 shows a pathway analysis of the lymphocyte-specific gene (A) or urine peptide (B) markers. Data mining software (Ingenuity Systems, CA) was used with differentially (KD vs. FC) expressed genes or peptides to identify gene ontology groups and relevant canonical signaling pathways. The intensity of the node color indicates the degree of up- or down-regulation in KD. Nodes are displayed using shapes that represent the functional classes of the gene products, and different line types represent various relationships. Relationships are primarily due to co-expression.

FIG. 6 shows a classification analysis. Misclassification errors are shown as a function of the threshold parameter.

FIG. 7 shows a density plot analysis of the Z-score (LAD and RCA) to quantify coronary artery lesions in KD patients.

FIG. 8 shows a schematic diagram of a diagnostic system.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D.M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a biomarker” includes a mixture of two or more biomarkers, and the like.

The term “about,” particularly in reference to a given quantity, is meant to encompass deviations of plus or minus five percent.

A “biomarker” in the context of the present invention refers to a biological compound, such as a polypeptide or polynucleotide which is differentially expressed in a sample taken from patients having KD as compared to a comparable sample taken from control subjects (e.g., a person with a negative diagnosis, normal or healthy subject). The biomarker can be a protein, a fragment of a protein, a peptide, or a polypeptide, or a nucleic acid, a fragment of a nucleic acid, a polynucleotide, or an oligonucleotide that can be detected and/or quantified. KD biomarkers include polypeptides comprising amino acid sequences from proteins including, but not limited to, collagen type 16 alpha 1 (COL16A1), collagen type 1 alpha 1 (COL1A1), collagen type 3 alpha 1 (COL3A1), uromodulin (UMOD), collagen type 9 alpha 3 (COL9A3), collagen type 23 alpha 1 (COL23A1), collectin sub-family member 12 (COLEC12), unnamed protein product Q6ZSL6 (Q6ZSL6), and EMI domain containing 1 (EMID 1); and peptide fragments thereof including, but not limited to, peptides comprising an amino acid sequence selected from the group consisting of SEQ ID NOS:1-13, or comprising an amino acid sequence displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto. KD biomarkers also include polynucleotides comprising nucleotide sequences from genes or RNA transcripts of genes, including but not limited to, TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, STAT1, CAMK4, MRPS25, P2RY8, ADD3, TRIM26, ARRB1, GNAS, ISG20, PCGF5, PRPF18, CRTAM, LHPP, RASGRP1, CMPK2, MS4A14, and RHOH.

The terms “polypeptide” and “protein” refer to a polymer of amino acid residues and are not limited to a minimum length. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

The phrase “differentially expressed” refers to differences in the quantity and/or the frequency of a biomarker present in a sample taken from patients having, for example, KD as compared to a control subject. For example, a biomarker can be a polypeptide or polynucleotide which is present at an elevated level or at a decreased level in samples of patients with KD compared to samples of control subjects. Alternatively, a biomarker can be a polypeptide or polynucleotide which is detected at a higher frequency or at a lower frequency in samples of patients with KD compared to samples of control subjects. A biomarker can be differentially present in terms of quantity, frequency or both.

A polypeptide or polynucleotide is differentially expressed between two samples if the amount of the polypeptide or polynucleotide in one sample is statistically significantly different from the amount of the polypeptide or polynucleotide in the other sample. For example, a polypeptide or polynucleotide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide or polynucleotide is differentially expressed in two sets of samples if the frequency of detecting the polypeptide or polynucleotide in samples of patients' suffering from KD, is statistically significantly higher or lower than in the control samples. For example, a polypeptide or polynucleotide is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents, including but not limited to, conditioned media resulting from the growth of cells and tissues in culture medium, e.g., recombinant cells, and cell components.

A “test amount” of a marker refers to an amount of a biomarker present in a sample being tested. A test amount can be either an absolute amount (e.g., g/ml) or a relative amount (e.g., relative intensity of signals).

A “diagnostic amount” of a biomarker refers to an amount of a biomarker in a subject's sample that is consistent with a diagnosis of KD. A diagnostic amount can be either an absolute amount (e.g., g/ml) or a relative amount (e.g., relative intensity of signals).

A “control amount” of a marker can be any amount or a range of amount which is to be compared against a test amount of a marker. For example, a control amount of a biomarker can be the amount of a biomarker in a person without KD. A control amount can be either in absolute amount (e.g., μg/ml) or a relative amount (e.g., relative intensity of signals).

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Immunoassay” is an assay that uses an antibody to specifically bind an antigen (e.g., a biomarker). The immunoassay is characterized by the use of specific binding properties of a particular antibody to isolate, target, and/or quantify the antigen. An immunoassay for a biomarker may utilize one antibody or several antibodies Immunoassay protocols may be based, for example, upon competition, direct reaction, or sandwich type assays using, for example, labeled antibody. The labels may be, for example, fluorescent, chemiluminescent, or radioactive.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a biomarker from specific species such as rat, mouse, or human can be selected to obtain only those polyclonal antibodies that are specifically immunoreactive with the biomarker and not with other proteins, except for polymorphic variants and alleles of the biomarker. This selection may be achieved by subtracting out antibodies that cross-react with biomarker molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Capture reagent” refers to a molecule or group of molecules that specifically bind to a specific target molecule or group of target molecules. For example, a capture reagent can comprise two or more antibodies each antibody having specificity for a separate target molecule. Capture reagents can be any combination of organic or inorganic chemicals, or biomolecules, and all fragments, analogs, homologs, conjugates, and derivatives thereof that can specifically bind a target molecule.

The capture reagent can comprise a single molecule that can form a complex with multiple targets, for example, a multimeric fusion protein with multiple binding sites for different targets. The capture reagent can comprise multiple molecules each having specificity for a different target, thereby resulting in multiple capture reagent-target complexes. In certain embodiments, the capture reagent is comprised of proteins, such as antibodies.

The capture reagent can be directly labeled with a detectable moiety. For example, an anti-biomarker antibody can be directly conjugated to a detectable moiety and used in the inventive methods, devices, and kits. In the alternative, detection of the capture reagent-biomarker complex can be by a secondary reagent that specifically binds to the biomarker or the capture reagent-biomarker complex. The secondary reagent can be any biomolecule, and is preferably an antibody. The secondary reagent is labeled with a detectable moiety. In some embodiments, the capture reagent or secondary reagent is coupled to biotin, and contacted with avidin or streptavidin having a detectable moiety tag.

“Detectable moieties” or “detectable labels” contemplated for use in the invention include, but are not limited to, radioisotopes, fluorescent dyes such as fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), Cyan Fluorescent Protein (CFP), Yellow Fluorescent Protein (YFP), Cerianthus Orange Fluorescent Protein (cOFP), alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hvgromycin-B-phosphotransferase (HPH), thymidine kinase (TK), lacZ (encoding .alpha.-galactosidase), and xanthine guanine phosphoribosyltransferase (XGPRT), Beta-Glucuronidase (gus), Placental Alkaline Phosphatase (PLAP), Secreted Embryonic Alkaline Phosphatase (SEAP), or Firefly or Bacterial Luciferase (LUC). Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“Substantially purified” refers to nucleic acid molecules or proteins that are removed from their natural environment and are isolated or separated, and are at least about 60% free, preferably about 75% free, and most preferably about 90% free, from other components with which they are naturally associated.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The invention relates to the use of biomarkers either alone or in combination with clinical parameters for diagnosis of KD. In particular, the inventors have discovered panels of biomarkers whose expression profiles can be used to diagnose KD and to distinguish KD from other inflammatory diseases, including infectious illness and acute febrile illness. The inventors have further developed a clinical scoring system for classifying patients according to their risk of having KD based on 7 clinical parameters (duration of fever, hemoglobin concentration, C-reactive protein concentration, white blood cell count, percent eosinophils, percent monocytes, and percent immature neutrophils). This clinical scoring system can be used alone or in combination with biomarker profiles in a sequential diagnostic method for determining appropriate treatment regimens for patients (see Example 1).

In order to further an understanding of the invention, a more detailed discussion is provided below regarding the identified biomarkers and clinical scoring system and methods of using them in prognosis, diagnosis, or monitoring treatment of KD.

Biomarkers

Biomarkers that can be used in the practice of the invention include polypeptides comprising amino acid sequences from proteins including, but not limited to, collagen type 16 alpha 1 (COL16A1), collagen type 1 alpha 1 (COL1A1), collagen type 3 alpha 1 (COL3A1), uromodulin (UMOD), collagen type 9 alpha 3 (COL9A3), collagen type 23 alpha 1 (COL23A1), collectin sub-family member 12 (COLEC12), unnamed protein product Q6ZSL6 (Q6ZSL6), and EMI domain containing 1 (EMID 1); and peptide fragments thereof including, but not limited to, peptides comprising amino acid sequences selected from the group consisting of SEQ ID NOS:1-13, or comprising amino acid sequences displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto. KD biomarkers also include polynucleotides comprising nucleotide sequences from genes or RNA transcripts of genes including, but not limited to, TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, STAT1, CAMK4, MRPS25, P2RY8, ADD3, TRIM26, ARRB1, GNAS, ISG20, PCGF5, PRPF18, CRTAM, LHPP, RASGRP1, CMPK2, MS4A14, and RHOH. Differential expression of these biomarkers is associated with KD and therefore expression profiles of these biomarkers are useful for diagnosing KD and distinguishing KD from other inflammatory conditions, including infectious illness and acute febrile illness.

Accordingly, in one aspect, the invention provides a method for diagnosing KD in a subject, comprising measuring the level of a plurality of biomarkers in a biological sample derived from a subject suspected of having KD, and analyzing the levels of the biomarkers and comparing with respective reference value ranges for the biomarkers, wherein differential expression of one or more biomarkers in the biological sample compared to one or more biomarkers in a control sample indicates that the subject has KD. When analyzing the levels of biomarkers in a biological sample, the reference value ranges used for comparison can represent the level of one or more biomarkers found in one or more samples of one or more subjects without KD (i.e., normal or control samples). Alternatively, the reference values can represent the level of one or more biomarkers found in one or more samples of one or more subjects with KD.

The biological sample obtained from the subject to be diagnosed is typically blood or urine, but can be any sample from bodily fluids, tissue or cells (e.g., blood cells, lymphocytes) that contain the expressed biomarkers. A “control” sample as used herein refers to a biological sample, such as blood, urine, tissue, or cells that are not diseased. That is, a control sample is obtained from a normal subject (e.g. an individual known to not have KD or any condition or symptom associated with the disease). A biological sample can be obtained from a subject by conventional techniques. For example, blood can be obtained by venipuncture; urine can be spontaneously voided by a subject or collected by bladder catheterization; and solid tissue samples can be obtained by surgical techniques according to methods well known in the art.

In certain embodiments, a panel of biomarkers is used for diagnosis of KD. Biomarker panels of any size can be used in the practice of the invention. Biomarker panels for diagnosing KD typically comprise at least 4 biomarkers and up to 30 biomarkers, including any number of biomarkers in between, such as 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the invention includes a biomarker panel comprising at least 4, or at least 5, or at least 6, or at least 7, or at least 8, or at least 9, or at least 10 or more biomarkers. Although smaller biomarker panels are usually more economical, larger biomarker panels (i.e., greater than 30 biomarkers) have the advantage of providing more detailed information and can also be used in the practice of the invention.

In another aspect, the invention includes a biomarker panel comprising a plurality of biomarkers for diagnosing KD, wherein one or more biomarkers are selected from the group consisting of a COL16A1 polypeptide, a COL1A1 polypeptide, a COL3A1 polypeptide, a UMOD polypeptide, a COL9A3 polypeptide, a COL23A1 polypeptide, a COLEC12 polypeptide, a Q6ZSL6 polypeptide, and an EMID1 polypeptide; or peptide fragments thereof; and a TLR7 polynucleotide, a CXCL10 polynucleotide, a LMO2 polynucleotide, a PLXDC1 polynucleotide, a MARCH1 polynucleotide, a IFI30 polynucleotide, a LYN polynucleotide, a CDC42EP2 polynucleotide, a MS4A14 polynucleotide, a PARP 14 polynucleotide, a RAC2 polynucleotide, a SRF polynucleotide, a NKTR polynucleotide, a LAP3 polynucleotide, a APOL3 polynucleotide, a STAT1 polynucleotide, a GCNT1 polynucleotide, a CAMK4 polynucleotide, a MRPS25 polynucleotide, a P2RY8 polynucleotide, a ADD3 polynucleotide, a TRIM26 polynucleotide, a ARRB1 polynucleotide, GNAS, a ISG20 polynucleotide, PCGF5, a PRPF 18 polynucleotide, a CRTAM polynucleotide, a LHPP polynucleotide, a RASGRP1 polynucleotide, a CMPK2 polynucleotide, and an RHOH polynucleotide. In one embodiment, the invention includes a biomarker panel comprising a COL16A1 polypeptide, a COL1A1 polypeptide, a COL3A1 polypeptide, a UMOD polypeptide, a COL9A3 polypeptide, a COL23A1 polypeptide, a COLEC12 polypeptide, a Q6ZSL6 polypeptide, and an EMID1 polypeptide; or peptide fragments thereof. An exemplary biomarker panel comprises 13 peptides consisting of sequences selected from the group consisting of SEQ ID NOS:1-13. In another embodiment, the invention includes a biomarker panel comprising a TLR7 polynucleotide, a CXCL10 polynucleotide, a LMO2 polynucleotide, a PLXDC1 polynucleotide, a MARCH1 polynucleotide, a IFI30 polynucleotide, a LYN polynucleotide, a CDC42EP2 polynucleotide, a MS4A14 polynucleotide, a PARP14 polynucleotide, a RAC2 polynucleotide, a SRF polynucleotide, a NKTR polynucleotide, a LAP3 polynucleotide, a APOL3 polynucleotide, a STAT1 polynucleotide, a GCNT1 polynucleotide, a CAMK4 polynucleotide, a MRPS25 polynucleotide, a P2RY8 polynucleotide, a ADD3 polynucleotide, a TRIM26 polynucleotide, a ARRB1 polynucleotide, GNAS, a ISG20 polynucleotide, PCGF5, a PRPF18 polynucleotide, a CRTAM polynucleotide, a LHPP polynucleotide, a RASGRP1 polynucleotide, a CMPK2 polynucleotide, and an RHOH polynucleotide. Biomarkers panels are useful for diagnosing KD and distinguishing KD disease from other inflammatory conditions, including infectious illness and acute febrile illness.

In certain embodiments, clinical parameters are used for diagnosis of KD, either alone or in combination with the biomarkers described herein. In one embodiment, the invention includes a method for determining a clinical score for a subject suspected of having KD. The method comprises measuring at least seven clinical parameters for the subject, including duration of fever, concentration of hemoglobin in the blood, concentration of C-reactive protein in the blood, white blood cell count, percent eosinophils in the blood, percent monocytes in the blood, and percent immature neutrophils in the blood. A clinical score can be calculated using, e.g., multivariate linear discriminant analysis (LDA) from the values of the clinical parameters. The clinical score can then be classified as a low risk KD clinical score, an intermediate risk KD clinical score, or a high risk KD clinical score by methods described herein (see Example 1).

A high risk KD clinical score or a low risk KD clinical score alone is sufficient to accurately diagnose a patient as either having KD or not having KD, respectively. For patients with intermediate risk KD clinical scores, additional information is needed to diagnose the patient accurately. A sequential diagnosis method can be used, wherein the clinical score information is combined with one or more biomarker profiles to diagnose the subject. Thus, in one embodiment, the invention includes a method for diagnosing KD in a subject comprising (i) determining a KD clinical score for the subject; and (ii) measuring the level of a plurality of biomarkers in a biological sample derived from the subject; and analyzing the levels of the biomarkers and comparing with respective reference value ranges for the biomarkers. For example, a panel of biomarkers comprising one or more COL16A1, COL1A1, COL3A1, UMOD, COL9A3, COL23A1, COLEC12, Q6ZSL6, and EMID1 polypeptides or peptide fragments thereof may be used in combination with the clinical score for diagnosis of KD. In one embodiment, the panel of biomarkers used in combination with the clinical store comprises peptides consisting of amino acid sequences selected from the group consisting of SEQ ID NOS:1-13, or comprising amino acid sequences displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto. Alternatively or in addition, a panel of biomarkers comprising one or more TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides can be used in combination with the clinical score for diagnosis of KD.

The methods described herein may be used to determine if a patient suspected of having KD should be treated with an intravenous immunoglobulin (WIG). A patients is selected for treatment with IVIG if the patient has a KD clinical score in the high risk range, or a KD clinical score in the intermediate risk range and a positive KD diagnosis based on the expression profile of one or more biomarker panels described herein.

Detecting and Measuring Levels of Biomarkers

It is understood that the expression level of the biomarkers in a sample can be determined by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites) that are indicative of the expression level of the biomarker. The methods for detecting biomarkers in a sample have many applications. For example, one or more biomarkers can be measured to aid in the diagnosis of KD, to determine the appropriate treatment for a subject, to monitor responses in a subject to treatment, or to identify therapeutic compounds that modulate expression of the biomarkers in vivo or in vitro.

Detecting Proteins, Polypeptides, and Peptides

In one embodiment, the expression levels of the biomarkers are determined by measuring protein, polypeptide, or peptide levels of the biomarkers. Assays based on the use of antibodies that specifically recognize the proteins, polypeptide fragments, or peptides of the biomarkers may be used for the measurement. Such assays include, but are not limited to, immunohistochemistry (IHC), western blotting, enzyme-linked immunosorbent assay (ELISA), radioimmunoassays (RIA), “sandwich” immunoassays, fluorescent immunoassays, immunoprecipitation assays, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 3142, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as ²H, ¹⁴C, ³²P, or ¹²⁵I, a fluorescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. and Cytochem., 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

It may be useful in the practice of the invention to fractionate biological samples, e.g., to enrich samples for lower abundance proteins to facilitate detection of biomarkers, or to partially purify biomarkers isolated from biological samples to generate specific antibodies to biomarkers. There are many ways to reduce the complexity of a sample based on the binding properties of the proteins in the sample, or the characteristics of the proteins in the sample.

In one embodiment, a sample can be fractionated according to the size of the proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of biomarkers.

In another embodiment, a sample can be fractionated by anion exchange chromatography. Anion exchange chromatography allows fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH are likely to be weakly negatively charged, and proteins that are eluted with an eluant having a low pH are likely to be strongly negatively charged. Thus, in addition to reducing complexity of a sample, anion exchange chromatography separates proteins according to their binding characteristics.

In yet another embodiment, a sample can be fractionated by heparin chromatography. Heparin chromatography allows fractionation of the biomarkers in a sample also on the basis of affinity interaction with heparin and charge characteristics. Heparin, a sulfated mucopolysaccharide, will bind biomarkers with positively charged moieties, and a sample can be sequentially eluted with eluants having different pH's or salt concentrations. Biomarkers eluted with an eluant having a low pH are more likely to be weakly positively charged. Biomarkers eluted with an eluant having a high pH are more likely to be strongly positively charged. Thus, heparin chromatography also reduces the complexity of a sample and separates biomarkers according to their binding characteristics.

In yet another embodiment, a sample can be fractionated by isolating proteins that have a specific characteristic, e.g. glycosylation. For example, a CSF sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Biomarkers that do not bind to the first adsorbent are removed with an eluant. The biomarkers that are in the fraction can be applied to a second adsorbent on the probe, and so forth. The advantage of performing sequential extraction on a gas phase ion spectrometer probe is that biomarkers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of the biomarkers if desired.

In yet another embodiment, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

Optionally, a biomarker can be modified before analysis to improve its resolution or to determine its identity. For example, the biomarkers may be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of biomarkers. The protein-binding molecules may be antibodies, peptides, peptoids, aptamers, small molecule ligands or other protein-binding capture agents attached to the surface of particles. Each protein-binding molecule may comprise a “unique detectable label,” which is uniquely coded such that it may be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes. See, e.g., U.S. Pat. No. 5,981,180, U.S. Pat. No. 7,445,844, U.S. Pat. No. 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp. 114-122; Nanobiotechnology Protocols Methods in Molecular Biology, 2005, Volume 303; herein incorporated by reference in their entireties).

In another example, biochips can be used for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by any appropriate means, for example, mass spectrometry, fluorescence, surface plasmon resonance, ellipsometry or atomic force microscopy.

Mass spectrometry, and particularly SELDI mass spectrometry, is a particularly useful method for detection of the biomarkers of this invention. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the biomarkers of this invention. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

Detecting Polynucleotides

In another embodiment, the expression levels of the biomarkers are determined by measuring polynucleotide levels of the biomarkers. The levels of transcripts of specific biomarker genes can be determined from the amount of mRNA, or polynucleotides derived therefrom, present in a biological sample. Polynucleotides can be detected and quantitated by a variety of methods including, but not limited to, microarray analysis, polymerase chain reaction (PCR), reverse transcriptase polymerase chain reaction (RT-PCR), Northern blot, and serial analysis of gene expression (SAGE). See, e.g., Draghici Data Analysis Tools for DNA Microarrays, Chapman and Hall/CRC, 2003; Simon et al. Design and Analysis of DNA Microarray Investigations, Springer, 2004; Real-Time PCR: Current Technology and Applications, Logan, Edwards, and Saunders eds., Caister Academic Press, 2009; Bustin A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008; herein incorporated by reference in their entireties.

In one embodiment, microarrays are used to measure the levels of biomarkers. An advantage of microarray analysis is that the expression of each of the biomarkers can be measured simultaneously, and microarrays can be specifically designed to provide a diagnostic expression profile for a particular disease or condition (e.g., Kawasaki disease).

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, the microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site.

Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm² and 25 cm²; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one embodiment, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids.

An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995); herein incorporated by reference in their entireties).

A second method for making microarrays produces high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270; herein incorporated by reference in their entireties) or other methods for rapid synthesis and deposition of defined oligonucleotides (Blanchard et al., Biosensors & Bioelectronics 11:687-690; herein incorporated by reference in its entirety). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684; herein incorporated by reference in its entirety), may also be used. In principle, any type of array, for example, dot blots on a nylon hybridization membrane (see Sambrook, et al., Molecular Cloning: A Laboratory Manual, 3rd Edition, 2001) could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123; herein incorporated by reference in their entireties. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink jetmethod are typically of high density, preferably having a density of at least about 2,500 different probes per 1 cm². The polynucleotide probes are attached to the support covalently at either the 3′ or the 5′ end of the polynucleotide.

Biomarker polynucleotides which may be measured by microarray analysis can be expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)⁺ messenger RNA (mRNA) or a fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. No. 5,545,522, 5,891,636, or 5,716,785). Methods for preparing total and poly(A)⁺ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation (Chirgwin et al., 1979, Biochemistry 18:5294-5299), a silica gel-based column (e.g., RNeasy (Qiagen, Valencia, Calif.) or StrataPrep (Stratagene, La Jolla, Calif.)), or using phenol and chloroform, as described in Ausubel et al., eds., 1989, Current Protocols In Molecular Biology, Vol. III, Green Publishing Associates, Inc., John Wiley & Sons, Inc., New York, at pp. 13.12.1-13.12.5). Poly(A)⁺RNA can be selected, e.g., by selection with oligo-dT cellulose or, alternatively, by oligo-dT primed reverse transcription of total cellular RNA. RNA can be fragmented by methods known in the art, e.g., by incubation with ZnCl₂, to generate fragments of RNA.

In one embodiment, total RNA, mRNA, or nucleic acids derived therefrom, are isolated from a sample taken from a KD patient. Biomarker polynucleotides that are poorly expressed in particular cells may be enriched using normalization techniques (Bonaldo et al., 1996, Genome Res. 6:791-806).

As described above, the biomarker polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one embodiment, biomarker polynucleotide molecules from a patient sample are labeled differentially from the corresponding polynucleotide molecules of a reference sample. The reference can comprise polynucleotide molecules from a normal biological sample (i.e., control sample, e.g., blood or urine from a subject not having KD) or from a KD reference biological sample, (e.g., blood or urine from a subject having KD).

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001), and in Ausubel et al., Current Protocols In Molecular Biology, vol. 2, Current Protocols Publishing, New York (1994). Typical hybridization conditions for the cDNA microarrays of Schena et al. are hybridization in 5.times.SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, Hybridization With Nucleic Acid Probes, Elsevier Science Publishers B. V.; and Kricka, 1992, Nonisotopic Dna Probe Techniques, Academic Press, San Diego, Calif. Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). Arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

In one embodiment, the invention includes a microarray comprising an oligonucleotide that hybridizes to a TLR7 polynucleotide, an oligonucleotide that hybridizes to a CXCL10 polynucleotide, an oligonucleotide that hybridizes to a LMO2 polynucleotide, an oligonucleotide that hybridizes to a PLXDC1 polynucleotide, an oligonucleotide that hybridizes to a MARCH1 polynucleotide, an oligonucleotide that hybridizes to a IFI30 polynucleotide, an oligonucleotide that hybridizes to a LYN polynucleotide, an oligonucleotide that hybridizes to a CDC42EP2 polynucleotide, an oligonucleotide that hybridizes to a MS4A14 polynucleotide, an oligonucleotide that hybridizes to a PARP14 polynucleotide, an oligonucleotide that hybridizes to a RAC2 polynucleotide, an oligonucleotide that hybridizes to a SRF polynucleotide, an oligonucleotide that hybridizes to a NKTR polynucleotide, an oligonucleotide that hybridizes to a LAP3 polynucleotide, an oligonucleotide that hybridizes to a APOL3 polynucleotide, an oligonucleotide that hybridizes to a STAT1 polynucleotide, an oligonucleotide that hybridizes to a GCNT1 polynucleotide, an oligonucleotide that hybridizes to a CAMK4 polynucleotide, an oligonucleotide that hybridizes to a MRPS25 polynucleotide, an oligonucleotide that hybridizes to a P2RY8 polynucleotide, an oligonucleotide that hybridizes to a ADD3 polynucleotide, an oligonucleotide that hybridizes to a TRIM26 polynucleotide, an oligonucleotide that hybridizes to a ARRB1 polynucleotide, an oligonucleotide that hybridizes to GNAS, an oligonucleotide that hybridizes to a ISG20 polynucleotide, an oligonucleotide that hybridizes to a PCGF5 polynucleotide, an oligonucleotide that hybridizes to a PRPF18 polynucleotide, an oligonucleotide that hybridizes to a CRTAM polynucleotide, an oligonucleotide that hybridizes to a LHPP polynucleotide, an oligonucleotide that hybridizes to a RASGRP1 polynucleotide, an oligonucleotide that hybridizes to a CMPK2 polynucleotide, and an oligonucleotide that hybridizes to an RHOH polynucleotide.

Polynucleotides can also be analyzed by other methods including, but not limited to, northern blotting, nuclease protection assays, RNA fingerprinting, polymerase chain reaction, ligase chain reaction, Qbeta replicase, isothermal amplification method, strand displacement amplification, transcription based amplification systems, nuclease protection (S1 nuclease or RNAse protection assays), SAGE as well as methods disclosed in International Publication Nos. WO 88/10315 and WO 89/06700, and International Applications Nos. PCT/US87/00880 and PCT/US89/01025; herein incorporated by reference in their entireties.

A standard Northern blot assay can be used to ascertain an RNA transcript size, identify alternatively spliced RNA transcripts, and the relative amounts of mRNA in a sample, in accordance with conventional Northern hybridization techniques known to those persons of ordinary skill in the art. In Northern blots, RNA samples are first separated by size by electrophoresis in an agarose gel under denaturing conditions. The RNA is then transferred to a membrane, cross-linked, and hybridized with a labeled probe. Nonisotopic or high specific activity radiolabeled probes can be used, including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radiolabelled cDNA, either containing the full-length, single stranded DNA or a fragment of that DNA sequence may be at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. Isotopes that can be used include, but are not limited to ³H, ¹⁴C, ³²P, ³⁵S, ³⁶Cl, ³⁵Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

Nuclease protection assays (including both ribonuclease protection assays and S1 nuclease assays) can be used to detect and quantitate specific mRNAs. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 g of sample RNA, compared with the 20-30 g maximum of blot hybridizations.

The ribonuclease protection assay, which is the most common type of nuclease protection assay, requires the use of RNA probes. Oligonucleotides and other single-stranded DNA probes can only be used in assays containing S1 nuclease. The single-stranded, antisense probe must typically be completely homologous to target RNA to prevent cleavage of the probe:target hybrid by nuclease.

Serial Analysis Gene Expression (SAGE), can also be used to determine RNA abundances in a cell sample. See, e.g., Velculescu et al., 1995, Science 270:484-7; Carulli, et al., 1998, Journal of Cellular Biochemistry Supplements 30/31:286-96; herein incorporated by reference in their entireties. SAGE analysis does not require a special device for detection, and is one of the preferable analytical methods for simultaneously detecting the expression of a large number of transcription products. First, poly A⁺ RNA is extracted from cells. Next, the RNA is converted into cDNA using a biotinylated oligo (dT) primer, and treated with a four-base recognizing restriction enzyme (Anchoring Enzyme: AE) resulting in AE-treated fragments containing a biotin group at their 3′ terminus Next, the AE-treated fragments are incubated with streptoavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a read-out of consecutive ditags of constant length. The presence of mRNA corresponding to each tag can then be identified from the nucleotide sequence of the clone and information on the sequence tags.

Quantitative reverse transcriptase PCR (qRT-PCR) can also be used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

A more recent variation of the RT-PCR technique is the real time quantitative PCR, which measures PCR product accumulation through a dual-labeled fluorigenic probe (i.e., TAQMAN probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Kits

In yet another aspect, the invention provides kits for diagnosing KD, wherein the kits can be used to detect the biomarkers of the present invention. For example, the kits can be used to detect any one or more of the biomarkers described herein, which are differentially expressed in samples of a KD patient and normal subjects. The kit may include one or more agents for detection of biomarkers, a container for holding a biological sample isolated from a human subject suspected of having KD; and printed instructions for reacting agents with the biological sample or a portion of the biological sample to detect the presence or amount of at least one KD biomarker in the biological sample. The agents may be packaged in separate containers. The kit may further comprise one or more control reference samples and reagents for performing an immunoassay or microarray analysis.

In certain embodiments, the kit comprises agents for measuring the levels of at least seven biomarkers of interest. For example, the kit may include agents for detecting biomarkers of a panel comprising COL16A1, COL1A1, COL3A1, UMOD, COL9A3, COL23A1, COLEC12, Q6ZSL6, and EMID1 polypeptides, or peptide fragments thereof. In one embodiment, the kit includes agents for detecting peptides of a biomarker panel comprising peptides consisting of sequences selected from the group consisting of SEQ ID NOS:1-13. In another embodiment, the kit includes agents for detecting polynucleotides of a biomarker panel comprising TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides. In addition, the kit may include agents for detecting more than one biomarker panel, such as two or three biomarker panels, which can be used alone or together in any combination, and/or in combination with clinical parameters for diagnosis of KD.

In one embodiment, the kit comprises at least one antibody selected from the group consisting of an antibody that specifically binds to a COL16A1 polypeptide, an antibody that specifically binds to a COL1A1 polypeptide, an antibody that specifically binds to a COL3A1 polypeptide, an antibody that specifically binds to a UMOD polypeptide, an antibody that specifically binds to a COL9A3 polypeptide, an antibody that specifically binds to a COL23A1 polypeptide, an antibody that specifically binds to a COLEC12 polypeptide, an antibody that specifically binds to a Q6ZSL6 polypeptide, and an antibody that specifically binds to an EMID1 polypeptide.

In another embodiment, the kit comprises at least one antibody selected from the group consisting of an antibody that specifically binds to a peptide comprising the amino acid sequence of SEQ ID NO:1, an antibody that specifically binds to a peptide comprising the amino acid sequence of SEQ ID NO:2, an antibody that specifically binds to a peptide comprising the amino acid sequence of SEQ ID NO:3, an antibody that specifically binds to a peptide comprising the amino acid sequence of SEQ ID NO:4, an antibody that specifically binds to a peptide comprising the amino acid sequence of SEQ ID NO:5, an antibody that specifically binds to a peptide comprising the amino acid sequence of SEQ ID NO:6, an antibody that specifically binds to a peptide comprising the amino acid sequence of SEQ ID NO:7, an antibody that specifically binds to a peptide comprising the amino acid sequence of SEQ ID NO:8, an antibody that specifically binds to a peptide comprising the amino acid sequence of SEQ ID NO:9, an antibody that specifically binds to a peptide comprising the amino acid sequence of SEQ ID NO:10, an antibody that specifically binds to a peptide comprising the amino acid sequence of SEQ ID NO:11, an antibody that specifically binds to a peptide comprising the amino acid sequence of SEQ ID NO:12, and an antibody that specifically binds to a peptide comprising the amino acid sequence of SEQ ID NO:13.

In another embodiment, the kit comprises a microarray for analysis of a plurality of biomarker polynucleotides. An exemplary microarray included in the kit comprises an oligonucleotide that hybridizes to a TLR7 polynucleotide, an oligonucleotide that hybridizes to a CXCL10 polynucleotide, an oligonucleotide that hybridizes to a LMO2 polynucleotide, an oligonucleotide that hybridizes to a PLXDC1 polynucleotide, an oligonucleotide that hybridizes to a MARCH1 polynucleotide, an oligonucleotide that hybridizes to a IFI30 polynucleotide, an oligonucleotide that hybridizes to a LYN polynucleotide, an oligonucleotide that hybridizes to a CDC42EP2 polynucleotide, an oligonucleotide that hybridizes to a MS4A14 polynucleotide, an oligonucleotide that hybridizes to a PARP14 polynucleotide, an oligonucleotide that hybridizes to a RAC2 polynucleotide, an oligonucleotide that hybridizes to a SRF polynucleotide, an oligonucleotide that hybridizes to a NKTR polynucleotide, an oligonucleotide that hybridizes to a LAP3 polynucleotide, an oligonucleotide that hybridizes to a APOL3 polynucleotide, an oligonucleotide that hybridizes to a STAT 1 polynucleotide, an oligonucleotide that hybridizes to a GCNT1 polynucleotide, an oligonucleotide that hybridizes to a CAMK4 polynucleotide, an oligonucleotide that hybridizes to a MRPS25 polynucleotide, an oligonucleotide that hybridizes to a P2RY8 polynucleotide, an oligonucleotide that hybridizes to a ADD3 polynucleotide, an oligonucleotide that hybridizes to a TRIM26 polynucleotide, an oligonucleotide that hybridizes to a ARRB1 polynucleotide, an oligonucleotide that hybridizes to GNAS, an oligonucleotide that hybridizes to a ISG20 polynucleotide, an oligonucleotide that hybridizes to a PCGF5 polynucleotide, an oligonucleotide that hybridizes to a PRPF 18 polynucleotide, an oligonucleotide that hybridizes to a CRTAM polynucleotide, an oligonucleotide that hybridizes to a LHPP polynucleotide, an oligonucleotide that hybridizes to a RASGRP 1 polynucleotide, an oligonucleotide that hybridizes to a CMPK2 polynucleotide, and an oligonucleotide that hybridizes to an RHOH polynucleotide.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing KD.

The kits of the invention have a number of applications. For example, the kits can be used to determine if a subject has KD or some other inflammatory condition arising, for example, from infectious illness or acute febrile illness. In another example, the kits can be used to determine if a patient should be treated with IVIG. In another example, kits can be used to monitor the effectiveness of treatment of a patient having KD. In a further example, the kits can be used to identify compounds that modulate expression of one or more of the biomarkers in in vitro or in vivo animal models to determine the effects of treatment.

Diagnostic System and Computerized Methods for Diagnosis of KD

In a further aspect, the invention includes a computer implemented method for diagnosing a patient suspected of having KD. The computer performs steps comprising: receiving inputted patient data; calculating a clinical score for the patient; classifying the clinical score as a low risk KD clinical score, an intermediate risk KD clinical score, or a high risk KD clinical score; analyzing the level of a plurality of biomarkers and comparing with respective reference value ranges for the biomarkers; calculating the likelihood that the patient has KD; and displaying information regarding the diagnosis of the patient. In certain embodiments, the inputted patient data comprises at least 7 clinical parameters selected from the group consisting of duration of fever, concentration of hemoglobin in the blood, concentration of C-reactive protein in the blood, white blood cell count, percent eosinophils in the blood, percent monocytes in the blood, and percent immature neutrophils in the blood. The inputted patient data may further comprise values for the levels of one or more polypeptide or peptide biomarkers in a biological sample from a patient, wherein the biomarkers are selected from the group consisting of a COL16A1 polypeptide, a COL1A1 polypeptide, a COL3A1 polypeptide, a UMOD polypeptide, a COL9A3 polypeptide, a COL23A1 polypeptide, a COLEC12 polypeptide, a Q6ZSL6 polypeptide, and an EMID1 polypeptide; and peptide fragments thereof. Alternatively or in addition, the inputted patient data may further comprise values for the levels of one or more polynucleotide biomarkers in a biological sample from a patient, wherein the polynucleotide biomarkers are selected from the group consisting of a TLR7 polynucleotide, a CXCL10 polynucleotide, a LMO2 polynucleotide, a PLXDC1 polynucleotide, a MARCH1 polynucleotide, a IFI30 polynucleotide, a LYN polynucleotide, a CDC42EP2 polynucleotide, a MS4A14 polynucleotide, a PARP14 polynucleotide, a RAC2 polynucleotide, a SRF polynucleotide, a NKTR polynucleotide, a LAP3 polynucleotide, a APOL3 polynucleotide, a STAT1 polynucleotide, a GCNT1 polynucleotide, a CAMK4 polynucleotide, a MRPS25 polynucleotide, a P2RY8 polynucleotide, a ADD3 polynucleotide, a TRIM26 polynucleotide, a ARRB1 polynucleotide, GNAS, a ISG20 polynucleotide, PCGF5, a PRPF18 polynucleotide, a CRTAM polynucleotide, a LHPP polynucleotide, a RASGRP 1 polynucleotide, a CMPK2 polynucleotide, and an RHOH polynucleotide. For example, the inputted patient data may comprise values for the levels of polypeptides, peptides, or polynucleotides in a biomarker panel comprising 7 or more biomarkers for diagnosing KD. In one embodiment, the inputted patient data may comprise values for the levels of polypeptides in a biomarker panel comprising one or more COL16A1, COL1A1, COL3A1, UMOD, COL9A3, COL23A1, COLEC12, Q6ZSL6, and EMID1 polypeptides; or peptide fragments thereof. For example, the inputted patient data may comprise values for the levels of peptides in a biomarker panel comprising peptides consisting of sequences selected from the group consisting of SEQ ID NOS:1-13, or comprising sequences displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99% sequence identity thereto. In another embodiment, the inputted patient data may comprise values for the levels of polynucleotides in a biomarker panel comprising one or more TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH polynucleotides. In a further embodiment, the inputted patient data may comprise values for more than one biomarker panel (e.g., two, three, or four biomarker panels) which may include biomarker polypeptides, peptides, and/or polynucleotides used in any combination.

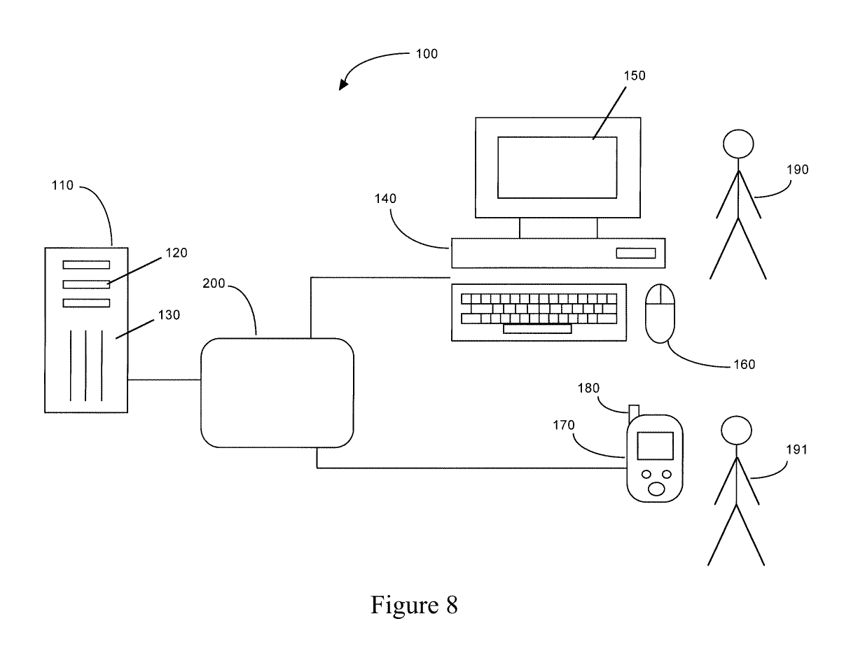

In a further aspect, the invention includes a diagnostic system for performing the computer implemented method, as described. As shown in FIG. 8, a diagnostic system 100 includes a computer 110 containing a processor 130, a storage component (i.e., memory) 120, a display component 150, and other components typically present in general purpose computers. The storage component 120 stores information accessible by the processor 130, including instructions that may be executed by the processor 130 and data that may be retrieved, manipulated or stored by the processor.

The storage component includes instructions for determining the diagnosis of the subject. For example, the storage component includes instructions for performing multivariate linear discriminant analysis (LDA), receiver operating characteristic (ROC) analysis, ensemble data mining methods, cell specific significance analysis of microarrays (csSAM), and multi-dimensional protein identification technology (MUDPIT) analysis and for performing a sequential diagnosis as described herein (see Example 1). The computer processor 130 is coupled to the storage component 120 and configured to execute the instructions stored in the storage component in order to receive patient data and analyze patient data according to one or more algorithms. The display component 150 displays information regarding the diagnosis of the patient.

The storage component 120 may be of any type capable of storing information accessible by the processor, such as a hard-drive, memory card, ROM, RAM, DVD, CD-ROM, USB Flash drive, write-capable, and read-only memories. The processor 130 may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor 130 in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel.

In one aspect, computer 110 is a server communicating with one or more client computers 140, 170. Each client computer may be configured similarly to the server 110, with a processor, storage component and instructions. Each client computer 140, 170 may be a personal computer, intended for use by a person 190-191, having all the internal components normally found in a personal computer such as a central processing unit (CPU), display 150 (for example, a monitor displaying information processed by the processor), CD-ROM, hard-drive, user input device (for example, a mouse, keyboard, touch-screen or microphone) 160, speakers, modem and/or network interface device (telephone, cable or otherwise) and all of the components used for connecting these elements to one another and permitting them to communicate (directly or indirectly) with one another. Moreover, computers in accordance with the systems and methods described herein may comprise any device capable of processing instructions and transmitting data to and from humans and other computers including network computers lacking local storage capability.

Although the client computers 140 and 170 may comprise a full-sized personal computer, many aspects of the system and method are particularly advantageous when used in connection with mobile devices capable of wirelessly exchanging data with a server over a network such as the Internet. For example, client computer 170 may be a wireless-enabled PDA such as a Blackberry phone, Apple iPhone, or other Internet-capable cellular phone. In such regard, the user may input information using a small keyboard, a keypad, a touch screen, or any other means of user input. The computer may have an antenna 180 for receiving a wireless signal.

The server 110 and client computers 140, 170 are capable of direct and indirect communication, such as over a network 200. Although only a few computers are depicted in FIG. 8, it should be appreciated that a typical system can include a large number of connected computers, with each different computer being at a different node of the network 200. The network, and intervening nodes, may comprise various combinations of devices and communication protocols including the Internet, World Wide Web, intranets, virtual private networks, wide area networks, local networks, cell phone networks, private networks using communication protocols proprietary to one or more companies, Ethernet, WiFi and HTTP. Such communication may be facilitated by any device capable of transmitting data to and from other computers, such as modems (e.g., dial-up or cable), networks and wireless interfaces. Server 110 may be a web server.

Although certain advantages are obtained when information is transmitted or received as noted above, other aspects of the system and method are not limited to any particular manner of transmission of information. For example, in some aspects, information may be sent via a medium such as a disk, tape, DVD, or CD-ROM. In other aspects, the information may be transmitted in a non-electronic format and manually entered into the system. Yet further, although some functions are indicated as taking place on a server and others on a client, various aspects of the system and method may be implemented by a single computer having a single processor.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Identifying Biomarkers for Kawasaki Disease (KD)

Patient Demographics and Sample Collection

Informed consent was obtained from the parents of all subjects and assent from all subjects greater than 6 years of age. This study was approved by the human subjects protection programs at the University of California San Diego (UCSD) and Stanford University. Inclusion criteria for KD subjects were based on the American Heart Association Guidelines (Newburger et al. (2004) Pediatrics, 114:1708-1733). All KD subjects had fever for at least three days and four of five classic criteria or three or fewer criteria with coronary artery abnormalities documented by echocardiogram. The 441 KD patients were distributed according to either the intravenous immunoglobulin (WIG) therapy outcome (Non responder: n=68; Responder: n=271; Late treatment: n=55; Non treated: n=16; IVIG+Remicade for coronary artery aneurysms: n=10; data not available: n=21) or the coronary artery lesion status (Normal: n=323; Aneurysms: n=34; Dilated: n=83; Data not available: n=1). Febrile control (FC) subjects were age-similar children evaluated for fever accompanied by at least one of the KD criteria (rash, conjunctival injection, oral mucosa changes, extremity changes, enlarged cervical lymph node). Febrile children with prominent respiratory or gastrointestinal symptoms were specifically excluded such that the majority of the controls had KD in the differential diagnosis of their condition. All subjects provided samples of blood and urine and underwent other diagnostic tests at the discretion of the managing clinicians. De-identified clinical laboratory test data were extracted from the UCSD KD electronic database for multivariate analysis. FC patients had a clinically or culture proven etiology for their febrile illnesses or underwent resolution of fever and clinical signs within three days of obtaining their clinical samples (designated as ‘viral syndrome’).

We compiled 3 cohorts of KD and FC subjects evaluated for their febrile illnesses at Rady Children's Hospital San Diego (Tables 1-4): 783 for clinical score development (clinical group: 441 KD and 342 FC); 106 for urine peptidome analysis (urine group: 53 KD and 53 FC); and 39 for cell type-specific microarray analysis of whole blood (blood group: 23 KD and 16 FC). The blood group (KD n=23, FC n=16) is a subset of previously analyzed samples (NCBI GEO GSE15297; Popper et al. (2009) J. Infect. Dis. 200:657-666; herein incorporated by reference in its entirety), peripheral whole blood expression analysis) with complete clinical data for all subjects. We chose KD and FC patients for the urine and blood groups with similar age and same gender. Patient demographic data were analyzed using SAS 9.2 (SAS Institute Inc., Cary, N.C., USA). The KD patients in the clinical group were more predominantly male and were younger than the FC patients but did not differ in ethnicity (Table 1). The 53 KD and 53 FC patients in the urine group were age-matched and did not differ in gender or ethnicity. Asian ethnicity was more common among KD subjects in the blood group.

KD clinical score calculation

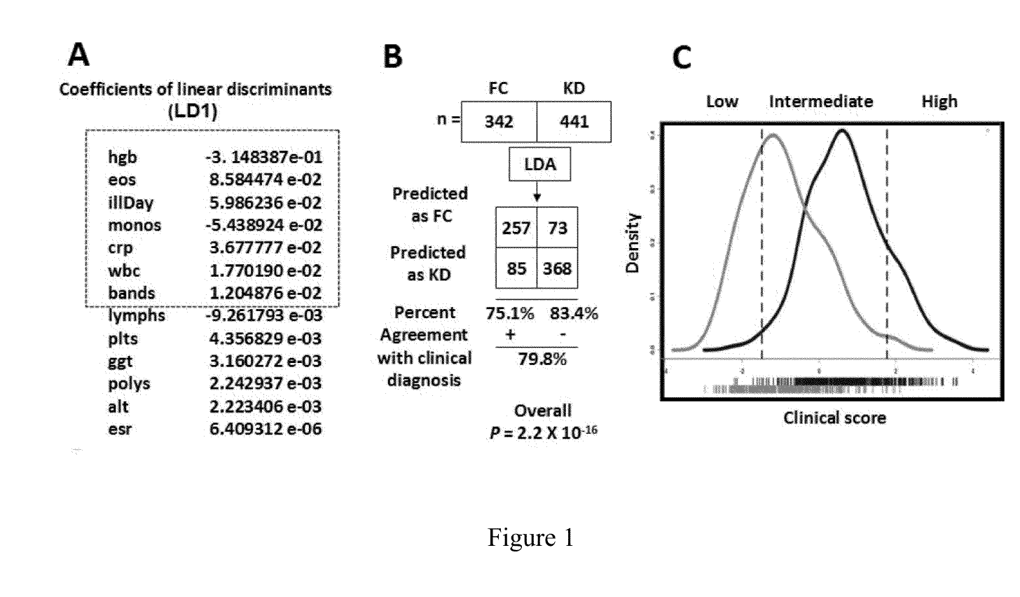

We used linear discriminant analysis (LDA) to stratify individual subjects based on a series of clinical exploratory variables. R (r-project.org/) library MASS function ‘lda’ was utilized. Coefficients of linear discriminants (LD1) were calculated as a measure of the association of each variable with the final diagnosis. The discriminant score was calculated from the seven variables (FIG. 1) with the largest (absolute value) coefficients. All patients were stratified into subgroups with low (5% likelihood KD), intermediate, and high (95% likelihood KD) clinical scores.

Microarray Analysis of Peripheral Whole Blood

We performed cell specific significance analysis of microarrays (csSAM) (Shen-Orr et al. (2010) Nat. Methods 7:287-289; herein incorporated by reference in its entirety) to analyze differential gene expression for each blood cell type in our previous KD array data set (NCBI GEO GSE15297; Popper, supra). Our expression analysis de-convoluted data from the major blood cell types: lymphocytes, neutrophils, immature neutrophils (band forms), monocytes, and eosinophils. For each gene, in each cell type, we calculated the contrast in its de-convoluted expression between KD and FC groups. The false discovery rate (FDR) was calculated as the ratio of genes whose differentiation exceeded a given threshold in the real dataset compared with the number of genes found significant by multiple permutations of the samples.

Urine Collection, Storage and Processing

Urine samples (5-10 mL) were either spontaneously voided or collected by bladder catheterization and held at 4° C. for up to 48 hours before centrifugation (2,000 g×20 minutes at room temperature) and freezing of the supernatant at −70° C. The details of urine processing, preparation of peptides, extraction and fractionation are reported elsewhere (Ling et al. (2010) Adv. Clin. Chem. 51:181-213; herein incorporated by reference in its entirety).

Urine Peptidomic Data Analysis

We pooled equal peptide content from 23 KD and 23 FC (Table 5) and subjected the pooled peptidome samples to multi-dimensional protein identification technology (MUDPIT), which uses strong cation exchange (SCX) and reverse phase (RP) chromatography and analysis using Fourier transform ion cyclotron resonance (FT ICR) mass spectrometry. The mass spectrometer's data-dependent acquisition isolates peptides as they elute and subjects them to Collision-Induced Dissociation, recording the fragment ions in a tandem mass spectrum. These spectra are matched to database peptide sequences by searching MS/MS (Mass spectrometry/Mass spectrometry) spectra against the Swiss-Prot database (version, Jun. 10, 2008) restricted to human entries (15,720 sequences) using the SEQUEST search engine. Searches were restricted to 50 and 100 ppm for parent and fragment ions, respectively. No enzyme restriction was selected. Since we were focusing on naturally occurring peptides, matches were considered significant when they were above the statistically significant threshold (as returned by SEQUEST BioWorks™ rev.3.3.1 SP1). Different fragmentation techniques were used for the validation of a peptide sequence, as well as for the detection, localization and characterization of the post-translational modifications. Due to the strong correlation between relative protein/peptide abundance and spectral counting summing all MS/MS spectra observed for the same peptide, the spectral counting method was used to compare the peptide abundance between KD and FC pooled samples. If the spectral counting of a peptide differed by two between KD and FC pooled samples, this peptide was chosen for ABI5800 matrix-assisted laser desorption/ionization (MALDI) TOF (Time of Flight) confirmation analysis. The individual peptidomes of 30 KD and 30 FC subjects (Table 6) were subjected to liquid chromatography-mass spectrometry (LCMS) based urine peptide profiling by ABI 5800. We targeted the 139 peptide biomarker candidates revealed by MUDPIT analysis and used their mass to charge ratio (m/z) values of the ions across all the LC fractions detected to construct extracted ion chromatograms (XICs) of individual urine samples. Windows for XIC construction were 25 ppm for m/z. Peak intensity values were normalized to the mean intensity of all peaks within a sample and then to the mean of the individual peptide ions across the samples. To follow up the potential peptide biomarkers, the statistical significance of each peptide's peak intensity between KD and FC groups was analyzed using the Mann Whitney U test and Student's t test. The urine peptide biomarker panel was analyzed by supporting vector machine (SVM) algorithm (R e1071 package). ROC analysis was performed (Zweig et al. (1993) Clin. Chem. 39:561-577; Sing et al. (2005) R. Bioinformatics 21:3940-3941; herein incorporated by reference in their entireties) to evaluate the performance of the clinical and molecular-based classifiers in the diagnosis of KD. Area under the ROC curve was calculated using the RORC package (Sing et al., supra).

Sequential Predictive Analysis Integrating Clinical and Molecular Findings for Diagnosis

To improve the diagnosis of patients with the intermediate clinical scores, we used Ensemble Data Mining Methods, also known as Committee Methods or Model Combiners (Oza NC: Ensemble data mining; 2006, NASA Ames Research Center. Moffett Field, Calif., USA; herein incorporated by reference in its entirety), to combine the clinical and molecular biomarker classifiers in order to derive practical algorithms for KD management. These machine learning methods combine the advantages of multiple models to achieve better predictive accuracy than is possible with any individual model (Oza, supra). We first stratified subjects into low, intermediate, and high risk groups based on clinical scores. Patients with intermediate KD clinical scores were further analyzed by either blood lymphocyte expression based or by urine peptidome based classifiers to improve diagnostic sensitivity and specificity.

Biological Pathway Analysis

Biological pathway analysis was performed with the Ingenuity IPA system (Ingenuity Systems, Redwood City, Calif.). To identify the canonical pathways that encompassed our KD biomarkers, 87 genes (94 significant probes) revealed by the cell type-specific gene expression studies of peripheral whole blood samples, and 13 significant urine peptide markers were mapped to known entries in the IPA canonical pathway database. The significance of the pathway was tested using Bioconductor (bioconductor.org) packages as previously described (Wu et al. (2009) Bioinformatics 25:832-833) and pathways with P-value <0.05 were chosen for further analysis.

Results

Development of KD Clinical Score

A data set of 783 patients, 342 FC and 441 KD, had complete records for 13 clinical and laboratory observations, which were used for exploratory multivariate linear discriminant analysis (LDA) (Tables 1-4): number of days of fever at time of clinical visit (illDay), total white blood cell (wbc), percentage monocytes (monos), lymphocytes (lymphs), eosinophils (eos), neutrophils (polys), immature neutrophils (bands), platelet counts (plts), hemoglobin (hgb), C-reactive protein (crp), gamma-glutamyl transferase (ggt), alanine aminotransferase (alt), and erythrocyte sedimentation rate (ESR). LDA created linear combinations of these clinical variables and calculated coefficients LD1 to optimize separation between KD and FC groups (FIG. 1). The discriminant model predicts clinical diagnosis with 79.8% overall accuracy (Fisher exact test P=2.2×10⁻¹⁶). The seven variables with the largest absolute values of coefficients LD1 were: days of illness, concentrations of hemoglobin and C-reactive protein, white blood cell count, and percentages of eosinophils, monocytes, and immature neutrophils for discriminant score calculation. The LDA discriminant scoring metric, designated as the KD “clinical score,” enables the seven clinical variables to be collectively interpreted on a scale, rather than a strict binary discrimination. Histograms of KD clinical scores demonstrate the distribution and considerable overlap of KD and FC patients (FIG. 1). Patients were stratified into three levels of risk for KD, determined by 95% correct classification effectiveness: low (clinical score <−1.48; 108 (96%) FC, (4%) KD), intermediate (−1.481≦clinical score ≦1.775, 366 KD and 230 FC) and high (clinical score >1.775; 70 (95%) KD, 4 (5%) FC) groups. Although the clinical score was accurate for subjects in the low (n=113) and high (n=74) clinical scoring groups, 596 patients (76%) had intermediate scores and remained unassigned by our clinical scoring algorithm.

Cell Type-Specific Significance Analysis (csSAM) of Peripheral Whole Blood Expression

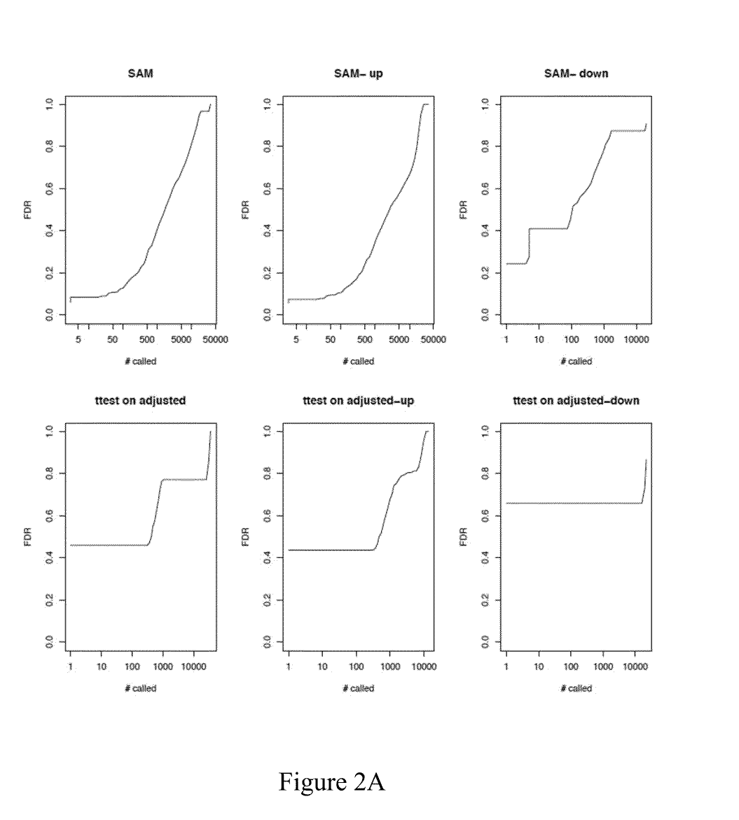

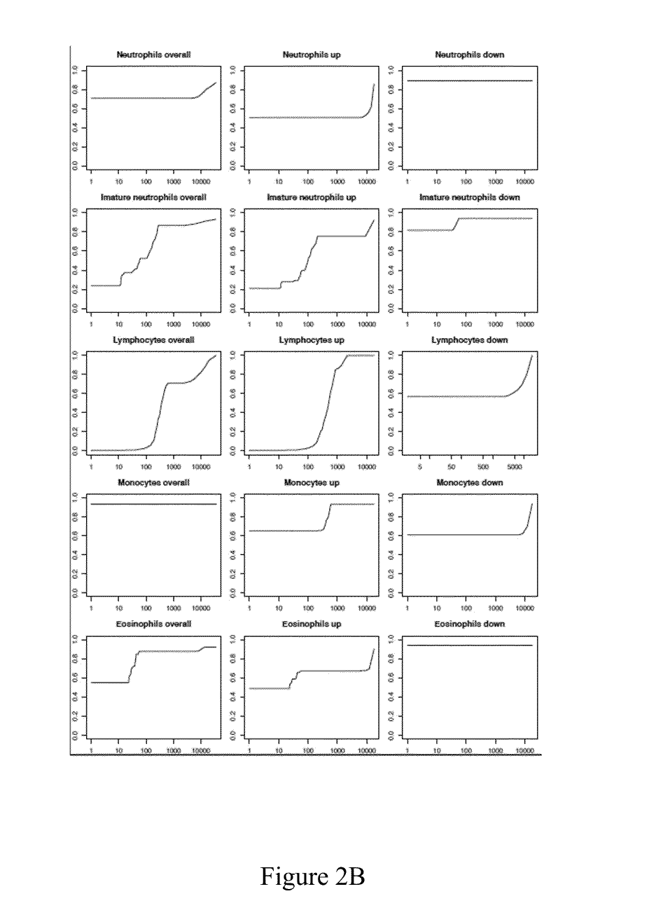

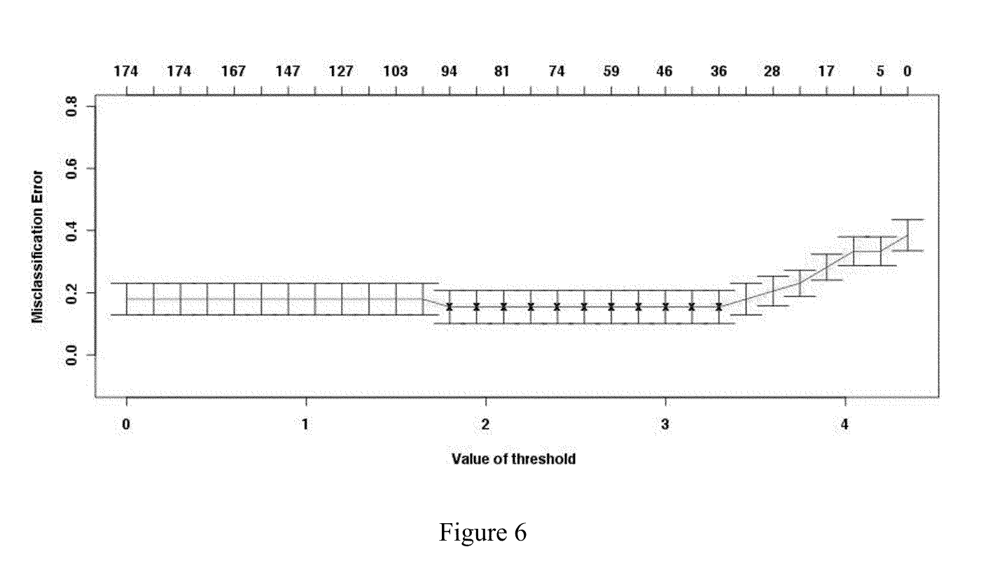

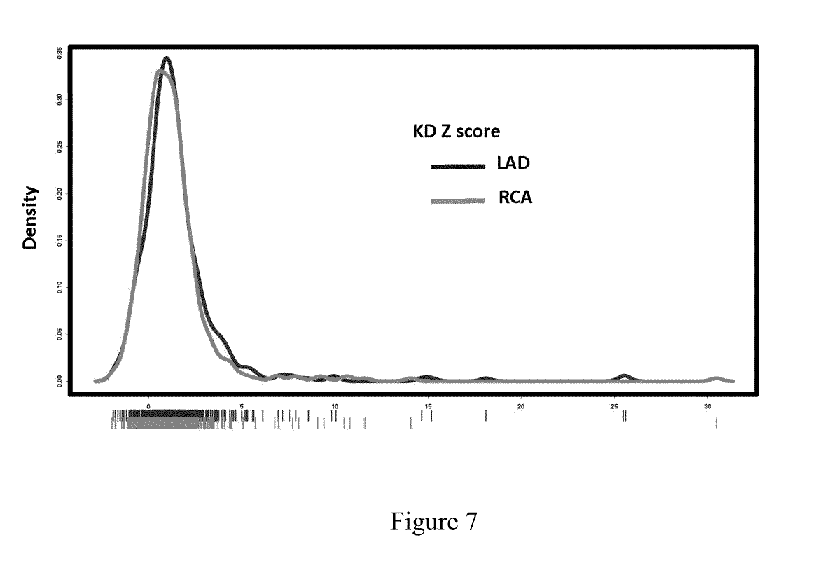

We employed the recently developed csSAM method (Shen-Orr et al. (2010) Nat. Methods, 7:287-289; herein incorporated by reference in its entirety), combining our KD array data set (NCBI GEO GSE15297; Popper et al, supra; blood testing cohort) and patients' relative cell type frequencies to analyze differential gene expression for each blood cell type in KD (n=23) and FC (n=16) subjects' whole blood. Whole-blood differential expression analysis using the Significance Analysis of Microarray (SAM) algorithm (Tusher et al. (2001) Proc. Natl. Acad. Sci. U.S.A. 98:5116-5121; herein incorporated by reference in its entirety), revealed no differentially expressed genes between the KD and FC groups at a relatively permissive FDR of 0.1 (FIG. 2A). For each of the KD and FC patients, we de-convoluted the cell type-specific gene expression profile, using csSAM, to perform cell type-specific differential expression analysis. Although the whole blood SAM analysis revealed no significant differentially expressed genes, and the lymphocyte count itself did not contribute to the clinical score, the csSAM analysis identified 87 differentially (down regulated in KD) expressed genes (94 gene probes; Table 6) in lymphocytes. Although eosinophil, monocyte and immature neutrophil relative counts had large LD1 coefficients for the KD clinical score, csSAM analysis identified no marker genes (FDR <0.05) for these blood cell types (FIG. 2B). FIG. 6 summarizes the training, 10 fold cross-validation, and test errors for different values of the threshold, revealing an effective KD/FC diagnostic gene marker panel (32 unique genes; top 36 gene probes in Table 7).

Urine Peptidome Analysis Discriminating KD and FC Patients

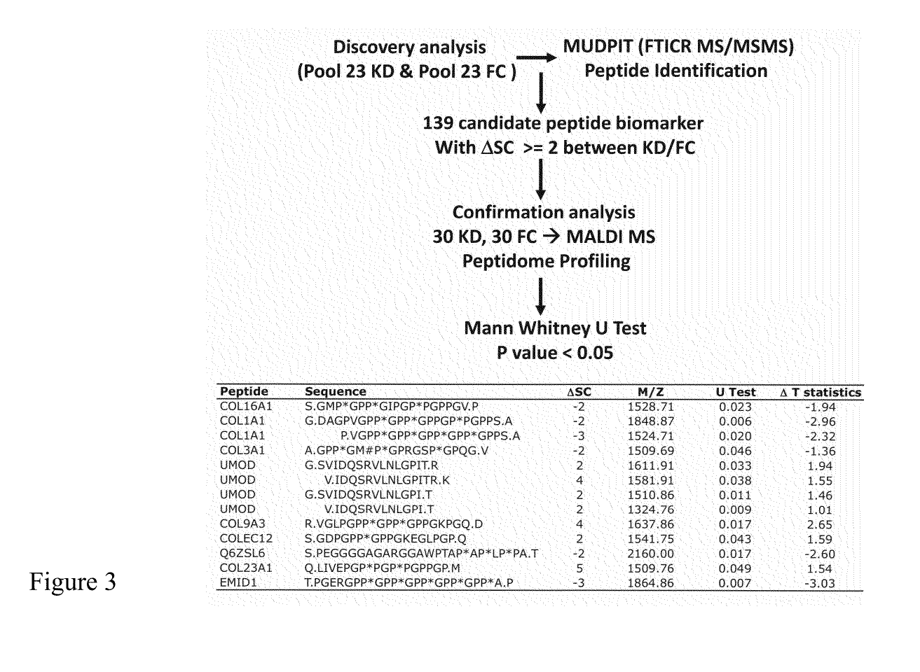

As shown in FIG. 3, for urine peptidome analyses, we have employed a combination of methods of multi-dimensional protein identification technology (MUDPIT: strong cation exchange SCX and reverse phase RP separations) analysis using Fourier transform ion cyclotron resonance (FT ICR) to discover candidate biomarkers in pooled KD (n=23) and FC (n=23) urine discovering cohort samples. Matrix-assisted laser desorption/ionization (MALDI) mass spectrometric (MS) TOF analysis was used to confirm these biomarkers in individual KD (n=30) and FC (n=30) urine testing cohort samples. Our exploratory MUDPIT analysis of pooled urine peptidomes yielded 139 candidate peptide biomarkers (FIG. 3). Subsequent MALDI TOF analysis confirmed the statistical significance of 13 urine peptides (FIG. 3), which are derived from 9 protein precursors (collagen type 16 alpha 1, collagen type 1 alpha 1, collagen type 3 alpha 1, uromodulin, collagen type 9 alpha 3, collagen type 23 alpha 1, collectin sub-family member 12, unnamed protein product Q6ZSL6, and EMI domain containing 1). Sequence alignment of these peptides revealed tight sequence clusters for the two COL1A1 and four UMOD peptides.

A Novel KD Diagnostic Algorithm Integrating Clinical and Molecular Biomarker Findings

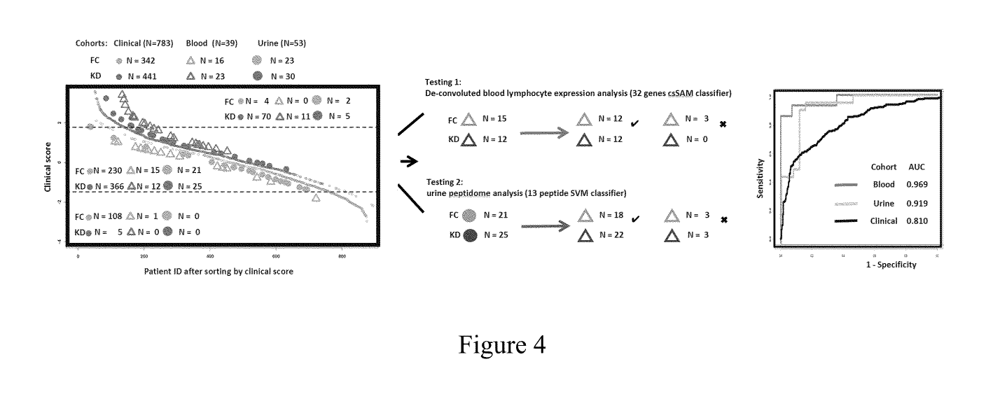

We first computed KD clinical scores for all patients in the clinical training, blood testing, and urine testing cohorts (FIG. 4, left panel). Although the clinical score had high sensitivity (blood: 11 of 11; urine: 5 of 5) and specificity (blood: 1 of 1; urine: 0 of 2) when limited to the low and high clinical score groups, the majority of the blood (27 of 39) and urine (46 of 53) testing cohorts were in the intermediate group where FC and KD patients had considerable clinical overlap. We applied gene expression or urine peptide based classifiers to better discriminate KD from FC subjects with intermediate KD clinical scores (FIG. 4, middle panel). The 32-lymphocyte-specific-gene-marker panel correctly classified 12 of 15 FC and 12 of 12 KD blood group patients with intermediate clinical scores. The 13-urine-peptide-biomarker panel correctly classified 18 of 21 FC and 22 of 25 KD urine group patients with intermediate clinical scores. ROC (FIG. 4, right panel) analysis revealed that molecular analyses of blood cell-specific gene expression (AUC 0.969) and of the urine peptidome (AUC 0.919) were superior to the clinical score (AUC 0.810) in differentiating KD from FC patients with intermediate clinical scores. This analysis suggests that the integration of clinical and molecular based panels provides an effective strategy for KD diagnosis. Febrile patients with low and high KD clinical scores are diagnosed with 95% confidence and need no further evaluation. Additional molecular-based testing, by either blood array profiling or urine peptidome analysis, refines the diagnostic performance for the remaining patients with intermediate clinical scores.

Biological Pathway Analyses of Blood Lymphocyte-Specific Gene Markers and Urine Peptide Biomarkers

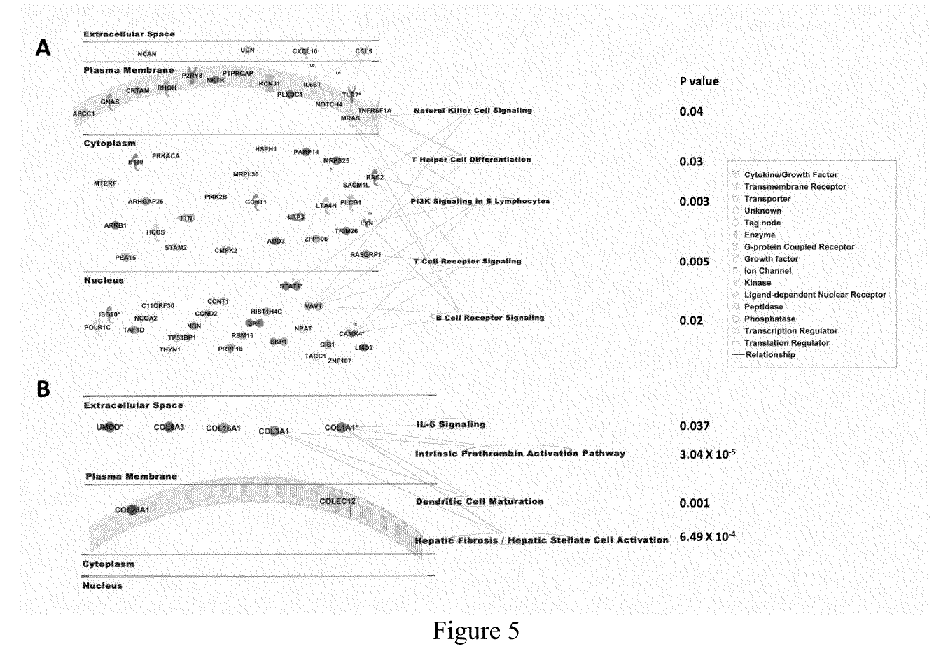

To characterize the canonical pathways in which our KD biomarkers are involved, 87 lymphocyte gene markers (94 significant probes) revealed by the cell type-specific expression of peripheral whole blood samples, and 13 confirmed urine peptide markers were mapped to known entries in the IPA (Ingenuity Pathway Analysis) canonical pathway database (FIG. 5). Cellular location analysis revealed that greater than 70% of the significant gene products reside within the cytoplasm and nucleus. In contrast, as expected, all of the significant urine peptides are derived from proteins located either in the extracellular space or on the plasma membrane. Pathway significance analysis (Wu et al. (2009) Bioinformatics 25:832-833; herein incorporated by reference in its entirety) of blood lymphocyte-specific gene markers revealed that PI3K signaling (P=0.003), T cell receptor signaling (P=0.005), B cell receptor signaling (P=0.02), T helper cell differentiation (P=0.03) and natural killer cell signaling (P=0.04) were significantly down-regulated in KD compared to FC patients. Urine peptidome pathway analysis revealed that the intrinsic prothrombin activation pathway (P=3.04×10⁻⁵), hepatic fibrosis/hepatic stellate cell activation (P=6.49×10⁻⁴), dendritic cell maturation (P=0.001), and IL-6 signaling (P=0.01) were significantly down-regulated in KD compared to FC.

Discussion

We have identified three different biomarker panels (7 clinical parameters, 32 blood lymphocyte-specific genes, 13 urine peptides) and developed an integrated algorithm to accurately diagnose KD. The clinical data we used in the multivariate analysis are routinely obtained during the evaluation of fever. However, clinicians have not used scoring systems derived by multivariate techniques for KD diagnosis. Although the clinical score correctly classified only 80% of febrile patients, patients with either low or high KD clinical scores were diagnosed as FC or KD respectively with 95% accuracy. For febrile patients with the confident diagnosis of KD, timely administration of WIG can thus be feasible to prevent the development of coronary artery dilatation or aneurysms. For febrile patients with intermediate clinical scores for whom confident diagnosis is not feasible, we developed a sequential algorithm, integrating clinical and molecular findings to improve KD diagnosis. Both the peripheral blood cell type-specific analysis and the urine peptidome biomarker analysis yielded sensitive and specific classifiers, which performed well in the diagnosis of KD. The csSAM-derived lymphocyte-specific gene markers and their mapped canonical pathways, for example PI3K signaling in B cells and T cell receptor signaling, provide insight into the host response in KD, and indicate that future research on the etiology of KD should focus on agents that suppress specific lymphocyte gene expression.

The overlapping sequences of the two COL1A1 and four UMOD peptides suggests that these peptide biomarkers reflect differential activities of disease-related proteases or their inhibitors such as TIMP 1 or matrix metalloproteinases in KD (Gavin et al. (2003) Arterioscler. Thromb. Vasc. Biol. 23:576-581; Lin et al. (2008) J. Orthop. Res. 26:1230-1237; Senzaki (2006) Arch. Dis. Child. 91:847-851; Peng et al. (2005) Zhonghua Er Ke Za Zhi 43:676-680; Chua et al. (2003) Pediatr. Nephroi. 18:319-327; Senzaki et al. (2001) Circulation 104:860-863; Matsuyama (1999) Pediatr. Int. 41:239-245). Serum peptide biomarker analysis of cancer subjects (Villanueva et al. (2006) J. Clin. Invest. 116:271-284) has demonstrated overlapping peptide biomarkers generated by disease-specific exo-peptidase activity. We have also observed tight clusters of urine peptide biomarkers in renal allograft dysfunction and SJIA (Ling et al. (2010) J. Am. Soc. Nephroi., 21:646-653). Therefore, the discovery of multiple overlapping collagen and uromodulin peptides suggests that the pathophysiology of KD involves the active degradation of proteins including collagen and uromodulin. With respect to the concern regarding incomplete KD cases hidden among the FC, we agree that inaccurate diagnosis is always one of the limitations in the absence of a gold standard diagnostic test. However, FC in this study included only patients whose illness resolved within three days of blood sampling or for whom a definite diagnosis was established (for example osteomyelitis, JIA). None of the FC included here had peeling in the convalescent phase. As for the KD patients, we have maintained a stable rate of coronary artery aneurysms from year to year (approximately 9%) suggesting that our diagnostic practices are stable. All the KD patients in this study were evaluated by one of two experienced clinicians at a single medical center. In this study, most of the FCs were enrolled by our team member, thus assuring consistency in diagnosis and sample collection. Our study is unique in focusing on a clinically relevant control group of children with fever who were actually being evaluated to rule in or rule out KD. All FC were evaluated with a standardized set of clinical laboratory tests that was also used to evaluate our KD patients. Our study also differs from many previous investigations on KD that used samples collected from a large number of hospitals that cared for only a few KD patients each. Therefore, a big problem with consistency in these studies was expected for comparative studies between KD and FC. Although all FC subjects in this study had laboratory testing for KD as recommended by the American Heart Association (AHA), very few FC had echocardiographic studies done. This is indeed a limitation. Although we acknowledge the potential inaccurate diagnosis of incomplete KD, our status as the sole freestanding children's hospital, sole KD referral center, and sole pediatric emergency department in San Diego County (catchment area of 5 million people) maximizes the likelihood that FC with persistent or progressive illness confused with KD would be captured during a return visit.

Our flexible clinical scoring metric is amenable to automation to develop data-driven predictive systems. Consistent with the current mandate to improve electronic medical record (EMR) use (Macaubas et al. (2010) Clin. Immunol. 134:206-216) and future interoperability between the hospital EMR and our predictive algorithm based applications consisting of demographic, clinical and genomic/proteomic data can serve an effective platform to allow interfacing between interdisciplinary teams (bed and bench side; what is known and what is practiced) for productive translational medicine.

### CONCLUSION

To the best of our knowledge, this is the first report describing a method integrating both clinical and molecular findings to discriminate KD from FC. Subsequent testing feedback from prospective KD/FC EMR data can be expected to further refine the clinical scoring metric and improve the KD diagnosis (Macaubas et al., supra).

While the preferred embodiments of the invention have been illustrated and described, it will be appreciated that various changes can be made therein without departing from the spirit and scope of the invention.

TABLE 1
Demographics of the 783 Patients
with Kawasaki Disease or Febrile Conditions

| | Kawasaki Disease | | Febrile Condition | | |
|---|---|---|---|---|---|
| | n = 441 (56.3%) | | n = 342 (43.7%) | | P-value |
| Age (months)^{a} | 35.6 | [1.0, 178.0, | 44.0 | [1.0, 210.0, | 0.002 |
| | | 29.7-33.9] | | 39.0-45.3] | |
| Male | 273 | (61.9%) | 190 | (55.6%) | 0.002 |
| Ethnicity | | | | | |
| Asian | 59 | (13.5%) | 31 | (9.1%), | 0.116 |
| African American | 17 | (3.9%) | 12 | (3.5%) | |
| Caucasian | 117 | (26.7%) | 87 | (25.5%) | |
| Hispanic | 149 | (34.0%) | 129 | (37.8%) | |
| Mixed | 90 | (20.6%) | 69 | (20.2%) | |

^{a}Reported as means with minimum, maximum, and 95% confidence interval in bracket. T-test was used. All other variables were reported as the number of patients and were analyzed using Fisher's Exact test.

TABLE 2
Demographics of the 106 Patients with Urine Peptidome Data

| | Kawasaki Disease | | Febrile Condition | | |
|---|---|---|---|---|---|
| | n = 53 (5.0%) | | n = 53 (50.0%) | | P-value |
| Age (months)^{a} | 50.6 | [3.0, 182.0, | 54.2 | [5.0, 209.0, | 0.670 |
| | | 33.6-49.8] | | 39.9-58.8] | |
| Male | 36 | (67.9%) | 33 | (62.3%) | 0.409 |
| Ethnicity | | | | | |
| Asian | 7 | (13.4%) | 6 | (11.3%) | 0.095 |
| African American | 1 | (1.9%) | 1 | (1.9%) | |
| Caucasian | 10 | (19.2%) | 12 | (22.6%) | |
| Hispanic | 18 | (34.6%) | 16 | (30.2%) | |
| Mixed | 16 | (30.8%) | 11 | (20.8%) | |

^{a}Reported as means with minimum, maximum, and 95% confidence interval in bracket. T-test was used. All other variables were reported as the number of patients and were analyzed using Fisher's Exact test.

TABLE 3
Demographics of the 39 Patients with Microarray Data

| | Kawasaki Disease | | Febrile Condition | | |
|---|---|---|---|---|---|
| | n = 23 (59.0%) | | n = 16 (41.0%) | | P-value |
| Age (months)^{a} | 43.2 | [5.0, 152.0, | 64.3 | [6.0, 188.0, | 0.125 |
| | | 27.1-49.6] | | 38.1-90.6] | |
| Male | 15 | (65.2%) | 9 | (69.2%) | 1.000 |
| Ethnicity | | | | | |
| Asian | 8 | (34.8%) | 0 | (0%) | 0.044 |
| African American | 1 | (4.4%) | 0 | (0%) | |
| Caucasian | 5 | (21.7%) | 7 | (43.8%) | |
| Hispanic | 6 | (26.1%) | 8 | (50.0%) | |
| Mixed | 3 | (13.0%) | 1 | (6.3%) | |

^{a}Reported as means with minimum, maximum, and 95% confidence interval in bracket. T-test was used. All other variables were reported as the number of patients and were analyzed using Fisher's Exact test.

TABLE 4
Demographics of the 441 Patients with Kawasaki Disease

| | | IVIG Therapy | | | | | | Coronary artery | | | |
|---|---|---|---|---|---|---|---|---|---|---|---|
| | | | | | | IVIG + | Data | | | | Data |
| KD | | Non- | | Late | Non | Remicade | not | | Aneu- | | not |
| Diagnosis^{a} | N = | responder | Responder | treatment | Treated | for CAA | available | Normal | rysms | Dilated | available |
| KD criteria | 6 | 0 | 4 | 0 | 0 | 0 | 2 | 2 | 2 | 2 | 0 |
| 1 TRUE | | | | | | | | | | | |
| AND | | | | | | | | | | | |
| KD criteria | | | | | | | | | | | |
| 2 TRUE | | | | | | | | | | | |
| KD criteria | 123 | 16 | 71 | 25 | 10 | 0 | 1 | 106 | 4 | 12 | 1 |
| 1 FALSE | | | | | | | | | | | |
| AND | | | | | | | | | | | |
| KD criteria | | | | | | | | | | | |
| 2 FALSE | | | | | | | | | | | |
| KD criteria | 306 | 52 | 192 | 30 | 6 | 10 | 16 | 215 | 26 | 65 | 0 |
| 1 TRUE | | | | | | | | | | | |
| AND KD | | | | | | | | | | | |
| criteria 2 | | | | | | | | | | | |
| FALSE | | | | | | | | | | | |
| KD criteria | 6 | 0 | 4 | 0 | 0 | 0 | 2 | 0 | 2 | 4 | 0 |
| 1 FALSE | | | | | | | | | | | |
| AND KD | | | | | | | | | | | |
| criteria 2 | | | | | | | | | | | |
| TRUE | | | | | | | | | | | |

^{a}Diagnostic KD criteria include rash; red eyes; oral mucosa changes such as red pharynx, red lips, red ‘strawberry’ tongue; extremity changes such as red, swollen hands/feets, peeling; enlarged cervical lymph node > 1.5 cm)
KD criteria 1: fever 3 or more days plus 4 or 5 diagnostic KD criteria
KD criteria 2: fever at least 5 days plus 2 or more diagnostic KD criteria plus coronary artery abnormality
CAA: coronary artery aneurysm

TABLE 5
Demographics of the 46 Patients with Peptidome Data
Analyzed by FTICR MUDPIT

| | Kawasaki Disease | | Febrile Condition | | |
|---|---|---|---|---|---|
| | n = 23 (50.0%) | | n = 23 (50.0%) | | P-value |
| Age (months)^{a} | 37.6 | [3.0, 94.0, | 47.6 | [8.0, 191.0, | 0.361 |
| | | 17.4-32.3] | | 35.9-65.8] | |
| Male | 17 | (73.9%) | 14 | (60.8%) | 0.189 |
| Ethnicity | | | | | |
| Asian | 3 | (13.6%) | 4 | (17.4%) | 0.866 |
| African American | 0 | (0%) | 1 | (4.4%) | |
| Caucasian | 6 | (27.3%) | 4 | (17.4%) | |
| Hispanic | 6 | (27.3%) | 8 | (34.8%) | |
| Mixed | 7 | (31.8%) | 6 | (26.1%) | |

^{a}Reported as means with minimum, maximum, and 95% confidence interval in bracket. T-test was used. All other variables were reported as the number of patients and were analyzed using Fisher's Exact test.

TABLE 6
Demographics of the 60 Patients with Peptidome
Data Profiled by MALDI 5800

| | Kawasaki Disease | | Febrile Condition | | |
|---|---|---|---|---|---|
| | n = 30 (50.0%) | | n = 30 (50.0%) | | P-value |
| Age (months)^{a} | 60.0 | [5.0, 182.0, | 59.3 | [5.0, 209.0, | 0.951 |
| | | 42.3-77.7] | | 41.1-77.4] | |
| Male | 19 | (63.3%) | 19 | (63.3%) | 1.000 |
| Ethnicity | | | | | |
| Asian | 4 | (13.3%) | 2 | (6.7%) | 0.025 |
| African American | 1 | (3.3%) | 0 | (0%) | |
| Caucasian | 4 | (13.3%) | 8 | (26.7%) | |
| Hispanic | 12 | (40.0%) | 8 | (26.7%) | |
| Mixed | 9 | (30.0%) | 5 | (16.7%) | |

^{a}Reported as means with minimum, maximum, and 95% confidence interval in bracket. T-test was used. All other variables were reported as number of patients and analyzed using Fisher's Exact test.

TABLE 7
Differentially expressed genes revealed by csSAM
analysis of whole blood expression data set.

NSC Value

| ID | Gene | 1-score | 2-score |
|---|---|---|---|
| 1 | TLR7 | −0.5752 | 0.8268 |
| 2 | CXCL10 | −0.5694 | 0.8185 |
| 3 | LMO2 | −0.5613 | 0.8068 |
| 4 | PLXDC1 | −0.5604 | 0.8056 |
| 5 | MARCH1 | −0.539 | 0.7748 |
| 6 | IFI30 | −0.5308 | 0.763 |
| 7 | LYN | −0.5264 | 0.7567 |
| 8 | CDC42EP2 | −0.5247 | 0.7542 |
| 9 | MS4A14 | −0.5239 | 0.7531 |
| 10 | PARP14 | −0.5189 | 0.746 |
| 11 | RAC2 | −0.5167 | 0.7428 |
| 12 | SRF | −0.496 | 0.713 |
| 13 | NKTR | −0.494 | 0.7101 |
| 14 | LAP3 | −0.4904 | 0.7049 |
| 15 | APOL3 | −0.4791 | 0.6887 |
| 16 | STAT1 | −0.4718 | 0.6782 |
| 17 | GCNT1 | −0.4667 | 0.6709 |
| 18 | CAMK4 | −0.4633 | 0.666 |
| 19 | STAT1 | −0.4471 | 0.6427 |
| 20 | CAMK4 | −0.4421 | 0.6356 |
| 21 | MRPS25 | −0.4169 | 0.5993 |
| 22 | P2RY8 | −0.4086 | 0.5874 |
| 23 | ADD3 | −0.3915 | 0.5629 |
| 24 | TRIM26 | −0.3915 | 0.5628 |
| 25 | ARRB1 | −0.3761 | 0.5406 |
| 26 | GNAS | −0.3676 | 0.5285 |
| 27 | ISG20 | −0.3635 | 0.5226 |
| 28 | PCGF5 | −0.3538 | 0.5086 |
| 29 | PRPF18 | −0.3506 | 0.504 |
| 30 | CRTAM | −0.3478 | 0.4999 |
| 31 | LHPP | −0.3438 | 0.4942 |
| 32 | RASGRP1 | −0.3393 | 0.4877 |
| 33 | CMPK2 | −0.3372 | 0.4847 |
| 34 | MS4A14 | −0.3345 | 0.4808 |
| 35 | TLR7 | −0.3341 | 0.4803 |
| 36 | RHOH | −0.3328 | 0.4784 |
| 37 | DTX4 | −0.3024 | 0.4347 |
| 38 | SACM1L | −0.2952 | 0.4244 |
| 39 | TLR7 | −0.2941 | 0.4227 |
| 40 | JOSD3 | −0.2917 | 0.4194 |
| 41 | ARHGAP26 | −0.2846 | 0.4091 |
| 42 | STAT1 | −0.2839 | 0.4082 |
| 43 | NBN | −0.2825 | 0.4061 |
| 44 | TTN | −0.279 | 0.4011 |
| 45 | SKP1 | −0.2733 | 0.3929 |
| 46 | PEA15 | −0.2716 | 0.3904 |
| 47 | ZFP106 | −0.262 | 0.3766 |
| 48 | SEZ6L | −0.2595 | 0.373 |
| 49 | CIB1 | −0.2512 | 0.3611 |
| 50 | HIST1H4C | −0.2489 | 0.3579 |
| 51 | KCNJ1 | −0.2486 | 0.3574 |
| 52 | LTA4H | −0.2405 | 0.3457 |
| 53 | TRIM56 | −0.2383 | 0.3426 |
| 54 | PLCB1 | −0.2191 | 0.315 |
| 55 | ABCC1 | −0.2162 | 0.3108 |
| 56 | PTPRCAP | −0.2135 | 0.3069 |
| 57 | CCL5 | −0.2095 | 0.3012 |
| 58 | VAV1 | −0.1987 | 0.2856 |
| 59 | RBM15 | −0.1956 | 0.2812 |
| 60 | LOC23117 | −0.1878 | 0.2699 |
| 61 | MALAT1 | −0.1873 | 0.2692 |
| 62 | HCCS | −0.1545 | 0.222 |
| 63 | C4orf41 | −0.1465 | 0.2105 |
| 64 | ISG20 | −0.1418 | 0.2039 |
| 65 | UCN | −0.133 | 0.1912 |
| 66 | TP53BP1 | −0.1318 | 0.1895 |
| 67 | NCAN | −0.1299 | 0.1867 |
| 68 | STAM2 | −0.1253 | 0.1801 |
| 69 | MRPL30 | −0.1235 | 0.1775 |
| 70 | CCNT1 | −0.1234 | 0.1774 |
| 71 | C11orf30 | −0.1231 | 0.177 |
| 72 | POLR1C | −0.1229 | 0.1766 |
| 73 | CCND2 | −0.1213 | 0.1744 |
| 74 | C1QTNF5 | −0.1205 | 0.1732 |
| 75 | HILS1 | −0.1184 | 0.1703 |
| 76 | TNFRSF1A | −0.1179 | 0.1695 |
| 77 | PRKACA | −0.1148 | 0.1651 |
| 78 | NPAT | −0.1125 | 0.1617 |
| 79 | MTERF | −0.1058 | 0.1522 |
| 80 | ATG16L2 | −0.1029 | 0.148 |
| 81 | SLAIN1 | −0.0935 | 0.1343 |
| 82 | IL6ST | −0.0918 | 0.1319 |
| 83 | MRAS | −0.0911 | 0.1309 |
| 84 | NOTCH4 | −0.0864 | 0.1242 |
| 85 | ZNF107 | −0.0834 | 0.1199 |
| 86 | THYN1 | −0.0819 | 0.1177 |
| 87 | NCOA2 | −0.081 | 0.1164 |
| 88 | TACC1 | −0.0749 | 0.1077 |
| 89 | PI4K2B | −0.0716 | 0.1029 |
| 90 | DMXL1 | −0.0711 | 0.1022 |
| 91 | HSPH1 | −0.066 | 0.0949 |
| 92 | C9orf78 | −0.0631 | 0.0907 |
| 93 | INTS9 | −0.0614 | 0.0882 |
| 94 | CYP2J2 | −0.0534 | 0.0767 |

1. A method for diagnosing Kawasaki disease (KD) in a subject, the method comprising:
(a) measuring the level of a plurality of biomarkers in a biological sample derived from the subject, wherein the plurality of biomarkers comprises:
(i) one or more polypeptides comprising an amino acid sequence from a protein selected from the group consisting of collagen type 16 alpha 1 (COL16A1), collagen type 1 alpha 1 (COL1A1), collagen type 3 alpha 1 (COL3A1), uromodulin (UMOD), collagen type 9 alpha 3 (COL9A3), collagen type 23 alpha 1 (COL23A1), collectin sub-family member 12 (COLEC12), unnamed protein product Q6ZSL6 (Q6ZSL6), and EMI domain containing 1 (EMID1); or peptide fragments thereof; or
(ii) one or more polynucleotides comprising a nucleotide sequence from a gene or an RNA transcript of a gene selected from the group consisting of TLR7, CXCL10, LMO2, PLXDC1, MARCH1, IFI30, LYN, CDC42EP2, MS4A14, PARP14, RAC2, SRF, NKTR, LAP3, APOL3, STAT1, GCNT1, CAMK4, MRPS25, P2RY8, ADD3, TRIM26, ARRB1, GNAS, ISG20, PCGF5, PRPF18, CRTAM, LHPP, RASGRP1, CMPK2, and RHOH; and
(b) analyzing the levels of the biomarkers in conjunction with respective reference value ranges for said plurality of biomarkers, wherein differential expression of one or more biomarkers in the biological sample compared to one or more biomarkers in a control sample from a normal subject indicates that the subject has KD.

2. The method of claim 1, further comprising distinguishing a diagnosis of KD from a diagnosis of infectious illness in the subject.

3. The method of claim 1, further comprising distinguishing a diagnosis of KD from a diagnosis of acute febrile illness in the subject.

4. The method of claim 1, wherein the plurality of biomarkers comprises one or more peptides comprising an amino acid sequence selected from the group consisting of SEQ ID NOS: 1-13.

5. The method of claim 1, wherein the plurality of biomarkers comprises one or more peptides comprising an amino acid sequence having at least 90% identity to an amino acid sequence selected from the group consisting of SEQ ID NOS: 1-13.

6. The method of claim 1, wherein the subject is a human being.

7. The method of claim 1, wherein measuring the level of the plurality of biomarkers comprises performing an enzyme-linked immunosorbent assay (ELISA), a radioimmunoassay (RIA), an immunofluorescent assay (IFA), immunohistochemistry (IHC), a sandwich assay, magnetic capture, microsphere capture, a Western Blot, surface enhanced Raman spectroscopy (SERS), flow cytometry, or mass spectrometry.

8. The method of claim 7, wherein measuring the level of a biomarker comprises contacting an antibody with the biomarker, wherein the antibody specifically binds to the biomarker, or a fragment thereof containing an antigenic determinant of the biomarker.

9. The method of claim 8, wherein the antibody is selected from the group consisting of a monoclonal antibody, a polyclonal antibody, a chimeric antibody, a recombinant fragment of an antibody, an Fab fragment, an Fab′ fragment, an F(ab′)₂ fragment, an Fᵥ fragment, and an scFᵥ fragment.

10. The method of claim 1, wherein measuring the level of the plurality of biomarkers comprises performing microarray analysis, polymerase chain reaction (PCR), reverse transcriptase polymerase chain reaction (RT-PCR), a Northern blot, or a serial analysis of gene expression (SAGE).

11. The method of claim 1, wherein the biological sample comprises blood cells.

12. The method of claim 11, wherein the biological sample comprises lymphocytes.

13. The method of claim 1, wherein the biological sample comprises urine.

14-24. (canceled)

25. A method of selecting a patient suspected of having KD for treatment with an intravenous immunoglobulin (IVIG), the method comprising: (a) determining the KD clinical score of the patient, and (b) selecting the patient for treatment with IVIG if the patient has a high risk KD clinical score; or an intermediate risk KD clinical score and a positive KD diagnosis based on the expression profile of a plurality of biomarkers, wherein the positive KD diagnosis is determined according to the method of claim 1.

26-59. (canceled)
